(19) United States
(12) Patent Application Publication
(10) Pub. No.: US 2022/0016079 A1
(43) Pub. Date: Jan. 20, 2022

(54) COMBINATION TREATMENT OF HIV INFECTIONS

(71) Applicant: Debiopharm International S.A., Lausanne (CH)

(72) Inventors: Grégoire Vuagniaux, Lausanne (CH); Norbert Wiedemann, Lausanne (CH)

(73) Assignee: Debiopharm International S.A., Lausanne (CH)

(21) Appl. No.: 17/296,462

(22) PCT Filed: Nov. 26, 2019
(86) PCT No.: PCT/EP2019/082624

§ 371 Date: May 24, 2021

(30) Foreign Application Priority Data
Nov. 26, 2018 (EP) 18208384.0
Nov. 30, 2018 (EP) 18209456.5

(51) Int. Cl. A61K 31/407; C07K 16/28; A61K 45/06; A61P 31/18

(52) U.S. Cl. CPC A61K 31/407; C07K 16/2818; A61K 2039/505; A61K 45/06; A61P 31/18; C07K 16/2827

(57) ABSTRACT

The present invention provides combination therapies for the treatment of HIV. The combination therapies of the present invention are aimed to reverse HIV latency and ultimately cure the disease. The combination therapies of the invention involve the use of an IAP inhibitor and the use of an immune checkpoint inhibitor.

### 1. TECHNICAL FIELD

The present invention pertains to the field of medicine and pharmaceutical sciences. More specifically, it provides drug compounds and compositions for combination therapy of HIV infections.

### 2. BACKGROUND OF THE INVENTION

HIV treatment involves taking medicines that slow the progression of the virus in the body. HIV is a type of virus called a retrovirus. At present, HIV patients are typically treated with combinations of 2 or more antiretroviral drugs (ARVs) having different viral targets. This therapeutic approach is termed combination antiretroviral therapy (ART). Typically employed in drug combinations comprise one or more of nucleoside/nucleotide reverse transcriptase inhibitors, non-nucleoside reverse transcriptase inhibitors, integrase inhibitors and/or protease inhibitors. More than 10 fixed-dose combinations of this type have received marketing authorization. Such fixed-dose drug combinations can achieve permanent suppression of HIV infection over many years, thereby greatly increasing life expectancy of HIV patients. Many of these ART drugs have been used since the mid-1990s and are the reason why the annual number of deaths related to AIDS has dropped over the past two decades.

The Office of AIDS Research Advisory Council (OARAC) of the US Department of Health and Human Services (HHS) emits Guidelines for the Use of Antiretroviral Agents in adults and Adolescents Living with HIV (https://aidsinfo.nih.gov/contentfiles/Ivguidelines/adultandadolescentgl.pdf) with the purpose to provide HIV care practitioners in the United States with recommendations based on current knowledge of antiretroviral drugs (ARVs). Similarly, in Europe, the European AIDS Clinical Society (EACS), having the mission to promote excellence in standards of care (SOC) in HIV infection and related co-infections, emits European Guidelines for treatment of HIV-positive adults in Europe (http://www.eacsociety.org/files/2018_guidelines-9.1-english.pdf).

To date HIV cannot be cured. ART reduces the amount of virus (or viral load) in the blood and body fluids. The virus persists in dormant CD4+ cells not producing any virus, forming latent reservoirs in various organs and causing relapse after arrest of conventional antiretroviral treatment due to some cells breaking their dormancy and reactivating viral replication. In the dormant state these reservoirs cannot be eliminated by current therapies as the infected cells do contain the HIV genome but do not produce any virus that would lead to cytopathic effects or elimination by the immune system. Therefore, to completely cure HIV, it is necessary to reverse the dormant state of infected CD4+ cells to make them susceptible to antiviral treatment.

By consequence, one important aspect of HIV cure research is the development of agents suitable for reversing HIV latency. Such agents are frequently referred to as latency reversing agents or LRAs. Different classes of agents are currently under investigation as LRAs, including histone deacetylase inhibitors, protein kinase C agonists, bromodomain inhibitors, and DNA methyltransferase inhibitors.

The inhibitors of apoptosis (IAP) proteins form a family of proteins that regulate programmed cell death. They may contribute to the survival of cancer cells. Hence, inhibitors of IAP proteins are currently under investigation as potentially useful in drugs for the treatment of cancer, typically in combination with another treatment that induces apoptosis. Said IAP inhibitors are also discussed as potentially useful drugs in the treatment of HIV infections. In particular, they are expected to reverse HIV latency by binding to the BIRC2 and BIRC3 members (Alternative name Cellular inhibitor of apoptosis 1 (cIAP-1) and 2 (cIAP-2) respectively) of the IAP protein family to thereby modulate NF-κB signaling and thus to stimulate HIV replication (Pache et al., Cell Host & Microbe 18, 345-353, 2015 (http://dx.doi.org/10.1016/j.chom.2015.08.009), Rasmussen et al., Curr Opin HIV AIDS, 2017 January; 12(1): 96-104. doi:10.1097/COH.0000000000000328, and Pache et al., WO 2015/187998 A). Further references to the possibility of using IAP inhibitors in HIV therapy are found in Stevenson et al., US2009/0010941, and Wang et al., WO 2008/128171.

Reversing latency alone is not sufficient for curing HIV infections. It is also necessary to eradicate the reactivated HIV-infected cells. This combined approach of reversing latency and killing the reactivated cells is sometimes referred to as the “shock and kill” approach.

Different classes of agents are discussed as being potentially suitable for the killing aspect: the drugs and drug combinations employed in conventional ART are likely to be useful in this respect. Toll-like receptor agonists are another class of agents that is discussed for this purpose.

It is known that HIV infected cells may exhibit increased levels of immune checkpoint molecules at their surfaces. This may help infected cells to evade destruction by the immune system, an effect sometimes referred to as T cell exhaustion. Immune checkpoint inhibitors (ICIs) are primarily investigated and used as a promising approach for the treatment of cancer, but they are also discussed as potentially useful agents in the shock and kill approach for curing HIV.

The above-mentioned approaches including immune checkpoint inhibitors, IAP inhibitors, histone deacetylase inhibitors, protein kinase C agonists, bromodomain inhibitors, DNA methyltransferase inhibitors, apoptosis inducers and Toll-like receptor agonists are all discussed in a review by Rasmussen et al. in Curr Opin HIV AIDS, 2017 January ; 12(1): 96-104. doi:10.1097/COH.0000000000000328.

The use of IAP Inhibitors for the treatment of HIV infections is described in US 2017/196879 A1 and in S.-I. Hattori et al., FRONTIERS IN MICROBIOLOGY, vol. 9, 2018, DOI: 10.3389/fmicb.2018.02022. The use of anti-PD-1 antibodies in the treatment of HIV infections is described in V. Velu et al., RETROVIROLOGY, BIOMED CENTRAL LTD., LONDON, GB, vol. 12, no. 1, 8 February 2015, page 14, DOI: 10.1186/812977-015-0144-X and in A. Serrao et al., ANNALS OF HEMATOLOGY, BERLIN, Del., vol. 98, no. 6, pages 1505-1506, DOI: 10.1007/800277-018-3541-0. The use of Debio 1143 together with an anti-PD-1 inhibitor in the treatment of cancer is described in A. Attinger, et al., retrieved from the Internet: URL:https://cancerres.aacrjournals.org/content/78/13Supplement/4703. The suitability of Debio 1143 as a latency reversal agent is described in M. Bobardt, et al. in PLOS ONE, https://doi.org/10.1371/journal.pone.0211746. This document was published after the priority date and before the filing date of the present application.

Despite such a multitude of promising alternative therapeutic approaches, there is a still no efficient cure for HIV infections available today.

### 3. SUMMARY OF THE INVENTION

The present invention therefore aims to solve the problems of the state of the art by providing more efficient therapies for the treatment and preferably cure of HIV infections. In particular, the present invention provides a combination product and combination therapy for the treatment of HIV infections, which shows excellent efficacy in terms of reducing the viral reservoirs and which at the same time shows excellent efficacy in terms of killing the HIV replicating cells that have been re-activated from the dormant state and thus contributes to the ultimate goal of curing HIV. Regarding the latter aspect of killing the HIV replicating cells, superior effects may be achieved in terms of amplitude and/or duration of the effect.

The present invention further provides pharmaceutical compositions suitable for use in the above-mentioned combination therapy to thereby accomplish the above-mentioned effects of superior efficacy in terms of reversing HIV latency and/or killing of revived HIV replicating cells and thus ultimately contribute to the desired curing of HIV.

The present invention also provides methods of treating HIV infected patients by means of the above-mentioned combination therapy. This aspect also involves the accomplishment of the above-mentioned benefits of superior efficacy in terms of reversal of HIV latency and/or killing of revived HIV replicating cells to thereby contribute to the ultimate goal of curing HIV.

The above-mentioned objectives are accomplished by the compounds, combination products, compositions, uses and methods specified in the appended claims.

### 4. DESCRIPTION OF FIGURES

FIG. 1: Graphical representation of results of HIV reactivation experiment described in Example 1, wherein Debio 1143 alone was administered at various concentrations to JLat 10.6-GFP Cells.

FIG. 2: Graphical representation of results of HIV reactivation experiment described in Example 1, wherein Debio 1143 was administered at various concentrations together with tenofovir disoproxil fumarate, emtricitabine and raltegravir to JLat 10.6-GFP Cells.

FIG. 3: Graphical representation of results of HIV reactivation experiment described in Example 1, wherein Debio 1143 alone was administered at various concentrations to 2D10 Cells.

FIG. 4: Graphical representation of results of HIV reactivation experiment described in Example 1, wherein Debio 1143 was administered at various concentrations together with tenofovir disoproxil fumarate, emtricitabine and raltegravir to 2D10 Cells.

FIG. 5: Graphical representation of results of HIV reactivation experiment described in Example 1, wherein Debio 1143 alone was administered at various concentrations to 5A8 Cells.

FIG. 6: Graphical representation of results of HIV reactivation experiment described in Example 1, wherein Debio 1143 was administered at various concentrations together with tenofovir disoproxil fumarate, emtricitabine and raltegravir to 5A8 Cells.

FIG. 7: Graphical representation of results of cytotoxicity experiment described in Example 2, wherein Debio 1143, a combination of Debio 1143 with ART, and a control compound are tested in the LDH Assay in JLat 10.6 GFP+cells for 3 days.

FIG. 8: Graphical representation of results of cytotoxicity experiment described in Example 2, wherein Debio 1143, a combination of Debio 1143 with ART, and a control compound are tested in the LDH Assay in primary CD4+ T-Lymphocytes for 3 days.

FIG. 9: Graphical representation of results of cytotoxicity experiment described in Example 2, wherein Debio 1143, a combination of Debio 1143 with ART, and a control compound are tested in JLat 10.6 GFP+ cells in the Celltiter Glo Assay for 3 days.

FIG. 10: Graphical representation of results of cytotoxicity experiment described in Example 2, wherein Debio 1143, a combination of Debio 1143 with ART, and a control compound are tested in CD4+ T-Lymphocytes in the Celltiter Glo Assay for 3 days.

FIG. 11: Graphical representation of results of HIV reactivation in resting CD4+ T-lymphocytes derived from HIV ART-treated patient #1 with Debio 1143 alone.

FIG. 12: Graphical representation of results of HIV reactivation in resting CD4+ T-lymphocytes derived from HIV ART-treated patient #1 with Debio 1143 in combination with ART.

FIG. 13: Graphical representation of results of HIV reactivation in resting CD4+ T-lymphocytes derived from HIV ART-treated patient #2 with Debio 1143 alone.

FIG. 14: Graphical representation of results of HIV reactivation in resting CD4+ T-lymphocytes derived from HIV ART-treated patient #2 with Debio 1143 in combination with ART.

FIG. 15: Graphical representation of results of HIV reactivation in 2D10 cells by Debio 1143 or other LRAs as single agents.

FIG. 16: Graphical representation of results of cytotoxicity by LDH assay in 2D10 cells treated with Debio 1143 or other LRAs as single agents.

FIG. 17: Graphical representation of results of HIV reactivation in 2D10 cells treated with Debio 1143 in combination with other LRAs.

FIG. 18: Graphical representation of results of effect of Debio 1143 on cIAP1 degradation and NF-kB modulation in HIV-1 latent 2D10 cell line (A), or 293T cell line (B), or CD4+ T-lymphocytes (C).

FIG. 19: Graphical representation of results of effect of Debio 1143 on the lysis of HIV-infected resting CD4+ T cells (rCD4) by CD8+ T cells and NK cells in 24 h co-culture

FIG. 20: Graphical representation of results of effect of Debio 1143 on the lysis of HIV-infected resting CD4+ T cells (rCD4) by CD8+ T cells and NK cells in 48 h co-culture

FIG. 21: Graphical representation of results of efficacy study in HIV-1 infected humanized BLT mice described in Example 4, wherein starting 12 weeks after infection Debio 1143 or Anti-PD-1 were administered alone or in combination for 4 weeks and viral blood titers determined weekly by qPCR detection of viral RNA. Black arrow indicates the treatment period. Mean values are displayed. N=8 mice per group.

FIG. 22: Graphical representation of results of efficacy study in HIV-1 infected humanized

BLT mice described in Example 4, wherein starting 12 weeks after infection Debio 1143 or Anti-PD-1 were administered alone or in combination for 4 weeks and the frequency of exhausted circulating CD8+ T cells determined weekly by detection of PD-1 using flow cytometry. Black arrow indicates the treatment period. Mean values are displayed. N=8 mice per group.

FIG. 23: Graphical representation of results of efficacy study in HIV-1 infected humanized BLT mice described in Example 4, wherein starting 12 weeks after infection Debio 1143 or Anti-PD-1 were administered alone or in combination for 4 weeks and 2 weeks after treatment completion the viral titers in CD4+ T cells isolated from various organs determined by qPCR detection of viral RNA. Mean values are displayed. N=5 mice per group.

### 5. DETAILED DESCRIPTION OF THE INVENTION

### 5.1. Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

References to internet pages are meant to be references to the specified pages in the version as accessible on Nov. 26, 2018. The content of these pages may be assessed via the revision history function in case of Wikipedia pages and otherwise via internet archives such as the wayback machine (accessible under https://archive.org/web/) or the like.

In some embodiments, the term “about” refers to a deviation of ±10% from the recited value. When the word “about” is used herein in reference to a number, it should be understood that still another embodiment of the invention includes that number not modified by the presence of the word “about”.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies). However, intact, i.e. non-fragmented, monoclonal antibodies are preferred.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch & Kinet, 1991. Annu Rev Immunol 9: 457-92.

The term “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

It is indicated in the summary above that the present invention provides combination therapies, combination products and uses thereof in methods of treatment of patients in need thereof. For the sake of simplicity and brevity, the present disclosure nevertheless sometimes refers to combination therapies of the invention only, or to combination products of the invention only, or the like. Unless the context dictates otherwise, all such indications should be understood as references to all aspects of the present invention (i.e. combination therapy, combination product, method of treatment with the combination product and any other uses or applications of the invention, as described herein).

The term “combination product” can refer to (i) a product comprised of two or more regulated components that are physically, chemically, or otherwise combined or mixed and produced as a single entity; (ii) two or more separate products packaged together in a single package or as a unit and comprised of drug and device products, device and biological products, or biological and drug products; (iii) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv) any investigational drug, device, or biological product packaged separately that according to its proposed labeling is for use only with another individually specified investigational drug, device, or biological product where both are required to achieve the intended use, indication, or effect.

“Combination therapy”, “combination treatment”, “in combination with”, “together with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. The disclosure below sometimes relies on expressions such as the “combination therapy of the present invention” or the like. Unless the context dictates otherwise, these indications should be understood as references not only to the described combination therapy but also as disclosures of the respective features in the context of the materials to be used for this purpose, i.e. the respective compounds for use in the specified manner, the resulting drug combinations for the specified use, including kits and combination products for the specified use. Of course, descriptions of the “combination therapy of the present invention” are also to be understood as descriptions of methods of treating HIV patients in need thereof.

The term “CTLA-4 antagonist” or “CTLA-4 inhibitor” refers to a substance that is capable of binding to CTLA-4 such that the function of CTLA-4 is blocked or at least reduced. This can be an antibody (i.e. an anti-CTLA-4 antibody) or a small molecule. An “anti-CTLA-4 antibody” means an antibody, or antigen binding fragment thereof, which binds to human CTLA-4 so as to disrupt the interaction of CTLA-4 with a human B7 receptor. After binding to B7, CTLA4 can inhibit the activation of mouse and human T cells, playing a negative regulating role in the activation of T cells. As used herein, unless specifically stated, said B7 refers to B7-1 and/or B7-2; and their specific protein sequences refer to the sequences known in the art. Reference can be made to the sequences disclosed in the literature or GenBank, e.g., B7-1 (CD80, NCBI Gene ID: 941), B7-2 (CD86, NCBI Gene ID: 942).

The term “Debio 1143”, “AT-406”, or “SM-406” refers to (5S,8S,10aR)-N-benzhydryl-5-((S)-2-(methylamino)propanamido)-3-(3-methylbutanoyl)-6-oxodecahydropyrrolo[1,2-a][1,5]diazocine-8-carboxamide (CAS Registry Number: 1071992-99-8) and/or pharmaceutically acceptable salts thereof. Preferably, the free base form of Debio 1143 is used in any aspect of the present invention. Its synthesis has been described previously (Cai et al., 2011. J Med Chem. 54(8):2714-26 and WO 2008/128171—Example 16). Analogues of Debio 1143 may for instance be considered to be compounds, which, for instance, contain at least 70%, preferably 80%, more preferably at least 90% of the atoms in the same positions that are present in Debio 1143 and/or which show at least 70%, preferably 80%, more preferably at least 90% of the effect on clAP1 of Debio 1143. This means that conservative substitutions are possible in analogues of Debio 1143. Likewise, further substituents may be incorporated as long as there is no significant effect on the activity as specified above.

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“HIV” is an acronym for human immunodeficiency virus. The present text uses the acronym HIV in the sense of its established meaning as described for instance in the Wikipedia entry “HIV”, version of Nov. 1, 2018, or by S. Lucas and A. M. Nelson in J Pathol. 2015 January; 235(2):229-41. doi: 10.1002/path.4449. In preferred embodiments of the present application, references to HIV should be understood as references to HIV-1, as discussed for instance by J. Hemelaar in Trends Mol Med. 2012 March; 18(3):182-92. doi: 10.1016/j.molmed.2011.12.001. Epub 2012 Jan. 11 and also by A. Engelman and P. Cherepanov in Nat Rev Microbiol. 2012 Mar. 16; 10(4):279-90. doi: 10.1038/nrmicro2747.

“HIV latency” characterizes the phenomenon that in patients treated with antiretroviral therapy (ART), viral reservoirs persist despite treatment and lead to rapid viral rebound when ART is interrupted. HIV latency is due to the integration of a DNA copy of the HIV RNA genome into the host cell DNA genome. At this stage, the cells are normally not susceptible to ART. HIV Latency is discussed for instance by M. S. Dahabieh et al. in Annu Rev Med. 2015; 66:407-21. doi: 10.1146/annurev-med-092112-152941 and references cited therein.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom & Winter, 1991. JMB. 227: 381; Marks et al., 1991. JMB. 222: 581). Also available for the preparation of human monoclonal antibodies are methods described in Cole et al., 1985. Monoclonal Antibodies and Cancer Therapy, Alan R. Liss, page 77; Boerner et al., 1991. J Immunol. 147(1): 86; van Dijk & van de Winkel, 2001. Curr Opin Pharmacol. 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al., 2006. PNAS USA. 103: 3557, regarding human antibodies generated via a human B-cell hybridoma technology.

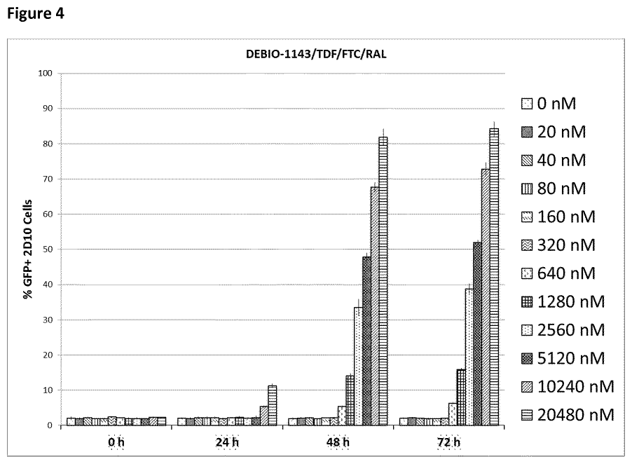

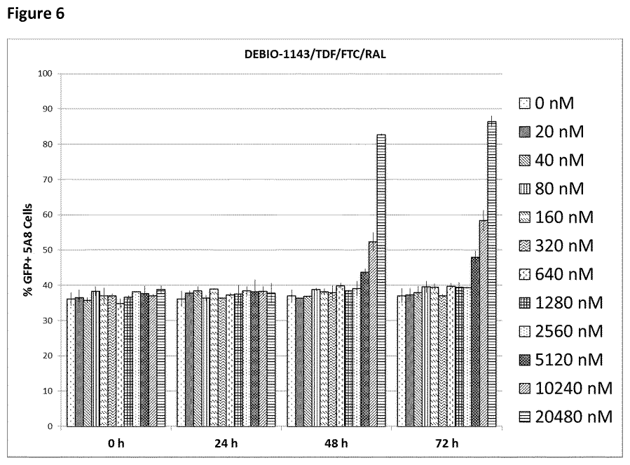

The term “IAP Inhibitor” is used herein to characterize a substance that is capable of inhibiting, blocking, slowing or reducing the function of IAP proteins. IAP proteins are proteins that regulate (inhibit) apoptosis. They are characterized by the presence of at least one BIR domain such as XIAP, cIAP1, cIAP2, Cp-IAP, NAIP, and Op-IAP. IAP proteins are described for instance in J. Silke and P. Meier, Cold Spring Harb Perspect Biol 2013; 5:a008730 and references cited therein. IAP inhibitors in the sense of the present invention are substances capable of inhibiting at least one of these IAP proteins, preferably two or more IAP proteins and most preferably cIAP1 and/or cIAP2. The Smac (Diablo) protein is an endogenous antagonist of IAP proteins. IAP inhibitors are therefore in some instances referred to as Smac mimetics. Such Smac mimetics are meant to be encompassed by the term “IAP inhibitor”. However, the present invention can also be successfully practiced with IAP inhibitors that are not Smac mimetics, e.g. because they have a clearly different structure. There is an interaction between IAP inhbitors and the BIR3 domain of IAP proteins. For the purpose of the present invention, it is of particular interest that an interaction between the IAP inhbitors and cIAP1 and/or cIAP2 leads to degradation of these proteins and subsequent NF-κB modulation. An example is given in example #6. In some embodiments, this effect can be used for testing a compound for IAP inhibitory activity: the experiment of example #6 is reproduced with the test compound. The effect is determined with a suitable technique including but not limited to western blot analysis of cells treated with the compound in vitro. For an IAP inhibitor, an effect on cIAP1 should be observed at concentrations below 10 μM, preferably <1 μM. An effect on cIAP1 may for instance be determined by means of the Western blot-based degradation experiment underlying FIG. 6 of Cai et al., 2011. J Med Chem. 54(8):2714-26. Alternatively, an IAP inhibitor may be identified as a compound having a Kᵢ of <1 μM against XIAP BIR3, cIAP1 BIR3 and/or cIAP2 BIR3, when carrying out the experiment underlying FIG. 4 of the above-mentioned publication by Cai et al.

The expression “Immune checkpoint inhibitor” (ICI) is used to specify a class of substances that interfere with the checkpoint mechanism of the immune system. This is a mechanism that modulates immune responses against own materials. In the context of therapy and especially cancer therapy, immune checkpoint inhibitors are a relatively new class of active compounds that amplify T-cell-mediated immune responses against cancer cells. The immune system relies on T cells to fight cancer. These specialized cells are extremely powerful and have the potential to damage healthy cells. T cell activity is controlled through “immune checkpoints,” which can be positive or negative. Positive immune checkpoints help T cells to continue their work, while negative immune checkpoints, such as CTLA-4 and PD-1, shut T cells off. In the context of the present invention, both inhibitory checkpoint molecules and stimulatory checkpoint molecules are targets of interest. Inhibitory checkpoint molecules and stimulatory checkpoint molecules are defined and described for instance in https://en.wikipedia.org/wiki/Immune checkpoint. Inhibitory checkpoint molecules include Programmed Death 1 receptor (PD-1) and its ligand (PD-L1), the Cytotoxic T-Lymphocyte-Associated protein 4 (CTLA-4), T cell immunoreceptor with Ig and ITIM domains (TIGIT), Lymphocyte Activation Gene-3 (LAG-3), T-cell Immunoglobulin domain and Mucin domain 3 (Tim-3), and any combination thereof. Inhibitors of such inhibitory checkpoint molecules include antibodies as well as small molecules. Such immune checkpoint inhibitors are described and discussed by N. Villanueva and L. Bazhenova in Ther Adv Respir Dis. 2018 January-December; 12: 1753466618794133, published online 2018 Sep. 14. doi: [10.1177/1753466618794133] and in the literature cited therein. The immune checkpoint inhibitor should exhibit high affinity binding to its target. Affinity is to be understood as the strength of the sum total of non-covalent interactions between a single binding site of the ICI and its binding partner. Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein, including Surface Plasmon Resonance (SPR, e.g. as analyzed on a BlAcore instrument) (Liljeblad et al., Glyco J 17, 323-329 (2000)), and traditional binding assays (Heeley, Endocr Res 28, 217-229 (2002). ICIs suitable for use in the present invention may have a dissociation constant (Kd) of ≤1 μM, ≤100 nM, ≤10 nM, ≤1 nM, ≤0.1 nM, ≤0.01 nM, or ≤0.001 nM (e.g. 10⁻⁷ M or less, e.g. from 10⁻⁷ M to 10⁻¹³ M, e.g. from 10⁻⁹ M to 10⁻¹³ M), with smaller dissociation constants being more preferred.

“Immunoglobulin” (Ig) is used interchangeably with “antibody” herein. In some embodiments, the basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the a and y chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins found in human serum: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

The terms “individual”, “patient” or “subject” are used interchangeably in the present application and are not meant to be limiting in any way. The “individual”, “patient” or “subject” can be of any age, sex and physical condition. Preferably, the methods of treatment and combination products of the present invention are for use in a human patient. In other words, the individual, patient or subject is preferably human.

“Infusion” or “infusing” refers to the introduction of a drug-containing solution into the body through a vein for therapeutic purposes. Generally, this is achieved via an intravenous bag.

A “patient in need thereof” in the context of the present invention is a patient infected with HIV. In some embodiments, it is a patient infected with HIV and preferably HIV-1, characterized by HIV infected CD4+ T cells exhibiting an increased level of PD-1, TIGIT, LAG-3 (as discussed by T. A. Rasmussen in Curr Opin HIV AIDS. 2017 January; 12(1): 96-104. doi:10.1097/COH.0000000000000328 and R. Fromentin et al. in PLOS Pathogens, Jul. 14, 2016, https://doLorg/10.1371/journal.ppat.1005761), CTLA-4 (as discussed by F. Wightman et al. in Curr Opin HIV AIDS. 2017 January; 12(1): 96-104. doi:10.1097/COH.0000000000000328) and/or other immune checkpoint molecules at their surfaces. In addition or alternatively, the patient may be characterized by an increased level of Tim-3 at the surfaces of the patient's CD8+ T cells (as described by R. B. Jones et al. in J. Exp. Med. Vol. 205 No. 12 2763-2779, www.jem.org/cgi/doi/10.1084/jem.20081398).

The term “PD-1” or “PD-1 receptor” refers to the programmed death-1 protein, a T-cell co-inhibitor, also known as CD279. The amino acid sequence of the human full-length PD-1 protein is set forth, for example, in GenBank Accession Number NP_005009.2. PD-1 is a 288 amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al., Immunol Rev, 2009, 229(1):356-386). The term “PD-1” includes recombinant PD-1 or a fragment thereof, or variants thereof. The PD-1 receptor has two ligands, PD-ligand-1 (PD-L1) and PD-ligand-2 (PD-L2).

The term “PD-1 inhibitor” refers to a substance that is capable of binding to the PD-1 receptor such that its immunomodulatory function is blocked completely or inhibited at least to a sufficient extent to make the substance useful as a therapeutic agent. For this purpose, the PD-1 inhibitor should have a binding affinity to its target as defined above with respect to the immune checkpoint inhibitors. The PD-1 inhibitor can be an antibody (anti-PD-1 antibody) or a small molecule. The term “anti-PD-1 antibody” or “an antibody that binds to PD-1” refers to an antibody that is capable of specifically binding PD-1 with sufficient affinity such that the antibody is useful as a therapeutic agent in targeting PD-1, or an antigen-binding fragment thereof that binds to PD-1 with sufficient affinity such that the fragment is useful as a therapeutic agent. Human PD-1 amino acid sequences can be found in NCBI Locus No.: NP 005009.

The term “PD-L1 inhibitor” refers to a substance that is capable of binding to the PD-L1 ligand such that its immunomodulatory function is blocked completely or inhibited at least to a sufficient extent to make the substance useful as a therapeutic agent. For this purpose, the PD-L1 inhibitor should have a binding affinity to its target as defined above with respect to the immune checkpoint inhibitors. The PD-L1 inhibitor can be an antibody (anti-PD-L1 antibody) or a small molecule. The term “anti-PD-L1 antibody” or “an antibody that binds to PD-L1” refers to an antibody that is capable of specifically binding PD-L1 with sufficient affinity such that the antibody is useful as a therapeutic agent in targeting PD-L1, or an antigen-binding fragment thereof that binds to PD-L1. Human PD-L1 amino acid sequence can be found in NCBI Locus No.: NP_054862.

The term “pharmaceutically acceptable adjuvant” refers to any and all substances which enhance the body's immune response to an antigen. Non-limiting examples of pharmaceutically acceptable adjuvants are: Alum, Freund's Incomplete Adjuvant, MF59, synthetic analogs of dsRNA such as poly(I:C), bacterial LPS, bacterial flagellin, imidazolquinolines, oligodeoxynucleotides containing specific CpG motifs, fragments of bacterial cell walls such as muramyl dipeptide and Quil-A®.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition. Pharmaceutical compositions comprising Debio 1143 preferably comprise Starch 1500 (reference to quality standard: Ph. Eur. 01/2010:1267) as a pharmaceutically acceptable excipient.

The term “pharmaceutically acceptable salts” is intended to include salts of the active compounds which are prepared with acids or bases, depending on the particular substituents found on the compounds described herein. When compounds of the present invention contain acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts. All references to active substance in the present application, including but not limited to IAP inhibitors and immune checkpoint inhibitors, are to be understood also as references to pharmaceutically acceptable salts of the respective specified active substances.

The expression “Stimulation of CD8+ T cells” is used herein to characterize an effect wherein the activity CD8+ effector immune cells is increased to thereby increase their ability to eliminate infected cells.

The term “therapeutically effective amount” refers to an amount of Debio 1143, and/or antibody or antigen-binding fragment thereof which has a therapeutic effect in the treatment of HIV infections. In particular, the therapeutically effective amount of the drug or drug combination leads to reversal of HIV latency and/or killing of HIV infected cells and preferably both of these therapeutic effects.

The terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

### 5.2. Overview

The present invention provides a combination therapy for treating HIV infections, including treatment methods as well as drugs, drug combinations, and kits for this use. This combination therapy relies on the use of at least one IAP inhibitor together with at least one immune checkpoint inhibitor. Unlike the established ART therapies, the present invention aims to cure HIV or at least reduce the patient's burden of latent HIV infected cells. To accomplish this objective, the present invention provides a combination of drugs that is capable of accomplishing a particularly beneficial profile of therapeutic effects:

1. Reversal of HIV latency: The data in the present application shows that the IAP inhibitor as single agent reactivates HIV transcription in cell lines and in blood from HIV-infected patients.
2. Enhanced immune response: The data in the present application shows that the IAP inhibitor, even when used as a single agent, stimulates CD8+ T cells to eliminate HIV-infected CD4+ T cells in vitro.
3. Enhanced immune response: The present data shows that HIV infection induces expression of exhaustion marker PD-1 in CD8+ T cells, hampering the immune system's capacity to eliminate the infection. Co-administration of at least one immune checkpoint inhibitor re-activates the exhausted CD8+ T cells.
4. Enhanced immune response: IAP inhibitor and ICI have complementary mechanisms on the immune system leading to enhanced immune response. The present data shows that the use of IAP inhibitors in combination with immune checkpoint inhibitors leads to an enhanced effect by both reactivating latent HIV and augmenting the anti-HIV immune response by CD8+ T cells.
5. Acceptable and/or manageable toxicity: The combination therapy of the present invention accomplishes its effects at acceptable and/or manageable levels of toxicity.
6. Enhanced reversal of HIV latency: Alternatively, if the efficacy of reversing HIV latency is to be further increased, the present invention permits to do so by co-administering a known latency reversing agent. The present data confirms that the combination of IAP inhibitor with a second latency reversing agent leads to a further enhancement of the latency reversing effect.

The present invention thus relies on the use of a specific drug combination that allows accomplishing a particularly favorable combination of individual therapeutic effects, which, in turn, leads to an enhanced efficacy in the cure of HIV.

The combination therapy of the present invention can be advantageously combined with the established ART treatments to thereby further enhance the treatment effect. This may include combinations of the combination therapy of the present invention with any one of the individual drug compounds used in ART, and it preferably includes also combinations of the combination therapy of the present invention with any one of the drug combinations employed in ART.

### 5.3. IAP Inhibitor

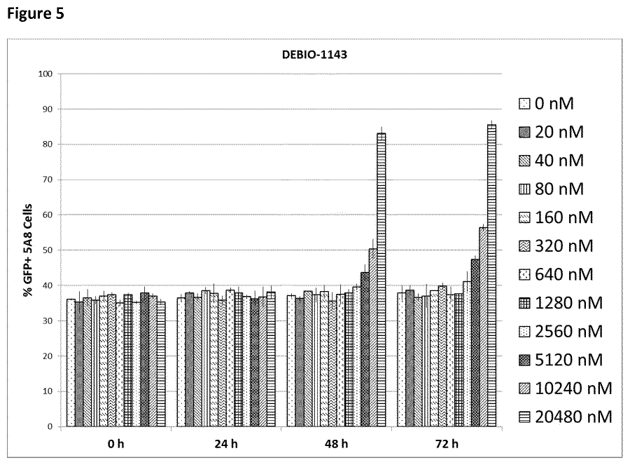

For the purpose of practicing the present invention, it is possible to use any compound that is capable of acting as an IAP inhibitor. This may include monovalent IAP antagonists such as Debio 1143 (Debiopharm, CAS No. 1071992-99-8), LCL-161 (Novartis, CAS No.: 1005342-46-0) and CUDC 427/GDC 0917 (Curis/Genentec, CAS No 1446182-94-0). Alternatively, bivalent IAP antagonists such as TL-32711/Birinapant (Medivir, CAS No.: 1260251-31-7), AZD5582 (AstraZeneca; CAS No. 1258392-53-8) and APG-1387 (Ascentage Pharma, SM-1387, CAS No. 1570231-89-8) may be used. Further useful IAP inhibitors include ASTX660 (Astex, CAS No. 1799328-86-1), SBP-0636457 (Sandford Burnham Prebys Medical Discovery Institute, CAS No. 1422180-49-1) and JP1201 (Joyant Pharmaceuticals), the structures of which are shown in FIGS. 5 and 6 of Finlay D, Teriete P, Vamos M et al. “Inducing death in tumor cells: roles of the inhibitor of apoptosis proteins” [version 1; referees: 3 approved]. F1000Research 2017, 6(F1000 Faculty Rev):587 (https://doLorg/10.12688/f1000research.10625.1). IAP antagonists, for which it is unknown whether they are mono- or bivalent are also suitable. This group includes IAP inhibitors developed by Boehringer Ingelheim (see in WO 2013/127729, WO 2015/025018, WO 2015/025019, WO 2016/023858, or WO 2018/178250), in particular IAP inhibitor called BI 891065). Of course, it is also possible in the present invention to use a combination of two or more different IAP inhibitors. In this case, each IAP inhibitor may be selected independently from the available IAP inhibitors as described herein.

Further suitable IAP Inhibitors are described for instance in WO 2008/128171 A, WO 2014/031487 A, WO 2011/050068 A, WO 2008/014240 A, WO 2007/131366 A, WO 2007/130626 A, WO 2011/057099, WO 2009/140447, EP 2 698 158, WO 2008/014229 A, WO 2017/117684 A1, WO 2016/079527 A1 and WO 2018/178250 A1 and also in Table 1 of WO 2017/143449 A, which refers to these compounds as Smac mimetic compounds. Yet another suitable IAP inhibitor is AZD5582 (AstraZeneca, CAS No. 1258392-53-8) as described WO 2010/142994 A1. All of such IAP inhibitors known from the literature may be used in the present invention.

The article by T. W. Owens et al. in J Carcinog Mutagen. 2013 May 27; Suppl 14: S14-004 (Published online 2013 May 27. doi: [10.4172/2157-2518.S14-004]) also provides information on suitable IAP inhibitors, especially in its Table 2 and literature cited therein.

It is of course also possible to use a combination of two or more IAP inhibitors. In this case, each IAP inhibitor may be independently selected.

### 5.4. Immune Checkpoint Inhibitor

In the present invention, any immune checkpoint inhibitor can be used, which is capable of inhibiting the immunomodulatory action of the immune checkpoints PD-1, PD-L1, CTLA-4, TIGIT, LAG-3, and Tim-3.

Suitable immune checkpoint inhibitors include those listed in Table 4 of WO 2017/143449 A1 and/or the immune checkpoint molecules described in WO 2016/054555 A2. The present invention also contemplates immune checkpoint inhibition at the DNA or RNA level as described on page 50 of WO 2016/054555 A2. Suitable immune checkpoint inhibitors are also discussed by M. J. Pianko in Stem Cell Investig. 2017; 4: 32, doi: [10.21037/sci.2017.03.04]. All immune checkpoint inhibitors known from the literature cited herein (and/or other literature) to be inhibitors of the above-mentioned immune checkpoint molecules may be used in the present invention.

In some embodiments of the present invention, the immune checkpoint inhibitor is selected from the group of PD-1 inhibitors and especially anti-PD-1 antibodies. In certain embodiments, the antibody or antigen-binding fragment thereof that binds to PD-1 includes, but is not limited to, pembrolizumab, nivolumab, spartalizumab, tislelizumab and pidilizumab. In some embodiments, the antibody or antigen-binding fragment thereof that binds to PD-1 is highly similar to pembrolizumab, nivolumab or pidilizumab and has no clinically meaningful differences with respect to safety and effectiveness as compared with the particular anti-PD-1 antibody. In some embodiments, the antibody or antigen-binding fragment thereof comprises an ADCC-competent Fc region. In particular, an anti-PD-1 antibody means an antibody that blocks or inhibits binding of PD-1 expressed on a HIV-infected cell to PD-Ll. In any of the treatment method, medicaments and uses of the present invention in which a human subject is being treated, the anti-PD-1 antibody specifically binds to human PD-1 and blocks or inhibits binding of human PD-L1 to human PD-1. The antibody may be a monoclonal antibody, human antibody, humanized antibody and/or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Such inhibitors are described for instance in WO 2016/054555 A, O. Hamid et al. New England Journal of Medicine 2013, 369(2): 134-44, WO 2009/114335, U.S. Pat. No. 8,609,089, US 2010/028330, US 2012/114649, U.S. Pat. Nos. 8,354,509, 7,521,051, 8,008,449, WO 2018/183408 A1 and WO2008/156712. The group of anti-PD-1 antibodies includes in particular Pembrolizumab, Pidilizumab, AMP514 (Medi0680, Amplimmune), REGN2810 (Regeneron) and Nivolumab. It also includes PD-1-binding fusion proteins as described for instance in WO 2010/027827 and WO 2011/066342. An example of an anti-PD-1 fusion protein is AMP-224 (Medlmmune, GSK), which is a recombinant B7-DC Fc-fusion protein composed of the extracellular domain of the PD-1 ligand programmed cell death ligand 2 (PD-L2, B7-DC) and the Fc region of human immunoglobulin (Ig) G1 (F. Smothers et al., Annals of Oncology, Volume 24, Issue suppl_1, 1 Mar. 2013, Pages i7, https://doi.org/10.1093/annonc/mdt042.6). Another possibility is to use a bispecific antibody as described for instance in US 2018/0326054.

In some embodiments of the present invention, the immune checkpoint inhibitor is selected from the group consisting of PD-L1 inhibitors and especially anti-PD-L1 antibodies and antigen-binding fragments thereof. In certain embodiments, the antibody or antigen-binding fragment thereof that binds to PD-L1 includes, but is not limited to, avelumab, atezolizumab, durvalumab, CX-072 (CytomX Therapeutics), BMS-936559 (MDX-1105, BMS). In some embodiments, the anti-PD-L1 antibody is avelumab (marketed in the United States under the Tradename Bavencio®). Avelumab is disclosed in International Patent Publication No. WO 2013/079174, the disclosure of which is hereby incorporated by reference in its entirety. Avelumab (formerly designated MSB0010718C) is a fully human monoclonal antibody of the immunoglobulin (Ig) G1 isotype (see e.g., WO 2013/079174). Avelumab selectively binds to PD-L1 and competitively blocks its interaction with PD-1. The mechanisms of action of avelumab rely on the inhibition of PD-1/PD-L1 interaction and on natural killer (NK)-based ADCC (see e.g., Boyerinas et al, 2015. Cancer Immunol Res. 3: 1148). In some embodiments, the antibody or antigen-binding fragment thereof that binds to PD-L1 is highly similar to avelumab, atezolizumab, durvalumab, CX-072 (CytomX Therapeutics), or BMS-936559 (MDX-1105, BMS) and has no clinically meaningful differences with respect to safety and effectiveness as compared with the particular anti-PD-L1 antibody. In some embodiments, the antibody or antigen-binding fragment thereof comprises an ADCC-competent Fc region. In particular, an anti-PD-L1 antibody means an antibody that blocks or inhibits binding of PD-1 expressed on a HIV-infected cell to PD-L1. In any of the treatment method, medicaments and uses of the present invention in which a human subject is being treated, the anti-PD-L1 antibody specifically binds to human PD-L1 and blocks or inhibits binding of human PD-L1 to human PD-1. The antibody may be a monoclonal antibody, human antibody, humanized antibody and/or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Examples of monoclonal antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, WO 2018/183408 A1 and U.S. Pat. Nos. 8,552,154, 8,779,108 and 8,383,796. Specific anti-human PD-L1 monoclonal antibodies useful as the PD-L1 antibody in the treatment method, medicaments and uses of the present invention include, for example without limitation, avelumab (MSB0010718C), MPDL3280A (an IgG1-engineered, anti-PD-L1 antibody), BMS-936559 (a fully human, anti-PD-L1, IgG4 monoclonal antibody), MED14736 (an engineered IgG1 kappa monoclonal antibody with triple mutations in the Fc domain to remove antibody-dependent, cell-mediated cytotoxic activity), and an antibody which comprises the heavy chain and light chain variable regions of SEQ ID NO:24 and SEQ ID NO:21, respectively, of WO 2013/019906. In some embodiments, the PD-L1 inhibitor may be a small molecule such as CA-170 (AUPM-170, Curis, Aurigene, described e.g. in J. J. Lee et al., Journal of Clinical Oncology 35, no. 15_suppl, DOI: 10.1200/JC0.2017.35.15_suppl.TPS3099). Further small molecule inhibitors of the PD-1/PD-L1 interaction, which are useful for the present invention, are described in WO 2018/195321 A.

In some embodiments of the present invention, the immune checkpoint inhibitor is selected from the group consisting of CTLA-4 inhibitors and especially anti-CTLA-4 antibodies and antigen-binding fragments thereof. In certain embodiments, the antibody or antigen-binding fragment thereof that binds to CTLA-4 includes, but is not limited to the human monoclonal antibody 10D1, now known as ipilimumab, and marketed as Yervoy™, as disclosed in U.S. Pat. No. 6,984,720. In another embodiment, the anti-CTLA-4 antibody is tremelimumab (CP-675,206), which is an IgG2 monoclonal antibody which is described in US 2012/263677, WO 2012/122444 or 2007/113648 A2. In further embodiments of the treatment methods, compositions and uses of the present invention, the anti-CTLA4 antibody, antigen binding fragment thereof, combination or variant thereof is as described in WO 2018/183408 A1 and WO 2018/035710 A1.

In some embodiments of the present invention, the immune checkpoint inhibitor is selected from the group consisting of inhibitors of T cell immunoreceptor with Ig and ITIM domains (TIGIT) and especially anti-TIGIT antibodies and antigen-binding fragments thereof. In certain embodiments, the antibody or antigen-binding fragment thereof that binds to TIGIT includes, but is not limited to, OMP-313M32 (mAb, OncoMed).

In some embodiments of the present invention, the immune checkpoint inhibitor is selected from the group consisting of inhibitors of Lymphocyte Activation Gene-3 (LAG-3), and especially anti-LAG-3 antibodies and antigen-binding fragments thereof. In certain embodiments, the antibody or antigen-binding fragment thereof that binds to LAG-3 includes, but is not limited to BMS-986016/Relatlimab (mAb Bristol Myers), LAG525 (mAb, Novartis), MGD013 (mAb, Macro-genics), REGN3767 (mAb, Regeneron Pharma), TSR-033 (mAb, Tesaro), and INCAGN022385 (mAb, Incyte Corp.).

In some embodiments of the present invention, the immune checkpoint inhibitor is selected from the group consisting of inhibitors of T-cell Immunoglobulin domain and Mucin domain 3 (Tim-3), and especially anti-Tim-3 antibodies and antigen-binding fragments thereof. In certain embodiments, the antibody or antigen-binding fragment thereof that binds to Tim-3 includes, but is not limited to LY3321367 (mAb, Eli Lilly and Company), MBG453 (mAb, Novartis), and TSR-022 (mAb, Tesaro).

It is of course also possible to use a combination of two or more immune checkpoint inhibitors. In this case, each immune checkpoint inhibitors may be independently selected. Such selections should involve especially two or more immune checkpoint inhibitors directed to two or more different target immune checkpoint molecules.

### 5.5. Combination Therapies

### Combinations of IAP Inhibitor and Immune Checkpoint Inhibitor

The present invention relies on the use of a combination of at least one IAP inhibitor with at least one immune checkpoint inhibitor active against PD-1, PD-L1, CTLA-4, TIGIT, LAG-3 and/or Tim-3. In some advantageous embodiments of the present invention, the combination therapy of the invention includes at least the following combinations of drugs:
(a) Debio 1143 or a Debio-1143 analogue in combination with an anti-PD-1 antibody;
(b) Debio 1143 or a Debio-1143 analogue in combination with an anti-PD-L1 antibody;
(c) Debio 1143 or a Debio-1143 analogue in combination with an anti-CTLA-4 antibody;
(d) Debio 1143 or a Debio-1143 analogue in combination with an anti-TIGIT antibody;
(e) Debio 1143 or a Debio-1143 analogue in combination with an anti-LAG-3 antibody;
(f) Debio 1143 or a Debio-1143 analogue in combination with an anti-Tim-3 antibody;
(g) Debio 1143 in combination with an anti-PD-1 antibody;
(h) Debio 1143 in combination with an anti-PD-L1 antibody;
(i) Debio 1143 in combination with an anti-CTLA-4 antibody;
(j) Debio 1143 in combination with an anti-TIGIT antibody;
(k) Debio 1143 in combination with an anti-LAG-3 antibody;
(l) Debio 1143 in combination with an anti-Tim-3 antibody;
(m) Debio 1143 in combination with an anti-PD-1 antibody selected from pembrolizumab, nivolumab, AMP514 (Medi0680, Amplimmune), REGN2810 (Regeneron), spartalizumab, tislelizumab and pidilizumab;
(n) Debio 1143 in combination with an with anti-PD-L1 antibody selected from avelumab, atezolizumab, durvalumab, CX-072 (CytomX Therapeutics), BMS-936559 (MDX-1105, BMS);
(o) Debio 1143 in combination with an with anti-CTLA-4 antibody selected from ipilimumab and tremelimumab;
(p) Debio 1143 in combination with an anti-TIGIT which is OMP-313M32 (mAb, OncoMed);
(q) Debio 1143 in combination with an with anti-LAG-3 antibody selected from BMS-986016 (mAb Bristol Myers), LAG525 (mAb, Novartis), MGD013 (mAb, Macro-genics), REGN3767 (mAb, Regeneron Pharma), TSR-033 (mAb, Tesaro), and INCAGN022385 (mAb, Incyte Corp.);
(r) Debio 1143 in combination with an with anti-Tim-3 antibody selected from LY3321367 (mAb, Eli Lilly and Company), MBG453 (mAb, Novartis), and TSR-022 (mAb, Tesaro);
(s) Debio 1143 in combination with any other therapy suggested or recommended in the OARAC Guidelines for the Use of Antiretroviral Agents in adults and Adolescents Living with HIV and/or EACS European Guidelines for treatment of HIV-positive adults in Europe.

Further preferred combinations involve the IAP inhibitor LCL 161 or analogues thereof.
(a′) LCL 161 or an LCL 161 analogue in combination with an anti-PD-1 antibody;
(b′) LCL 161 or an LCL 161 analogue in combination with an anti-PD-L1 antibody;
(c′) LCL 161 or an LCL 161 analogue in combination with an anti-CTLA-4 antibody;
(d′) LCL 161 or an LCL 161 analogue in combination with an anti-TIGIT antibody;
(e′) LCL 161 or an LCL 161 analogue in combination with an anti-LAG-3 antibody;
(f′) LCL 161 or an LCL 161 analogue or a Debio-1143 analogue in combination with an anti-Tim-3 antibody;
(g′) LCL 161 in combination with an anti-PD-1 antibody;
(h′) LCL 161 in combination with an anti-PD-L1 antibody;
(i′) LCL 161 in combination with an anti-CTLA-4 antibody;
(j′) LCL 161 in combination with an anti-TIGIT antibody;
(k′) LCL 161 in combination with an anti-LAG-3 antibody;
(l′) LCL 161 in combination with an anti-Tim-3 antibody;
(m′) LCL 161 in combination with an anti-PD-1 antibody selected from pembrolizumab, nivolumab, AMP514 (Medi0680, Amplimmune), REGN2810 (Regeneron), spartalizumab, tislelizumab and pidilizumab;
(n′) LCL 161 in combination with an anti-PD-L1 antibody selected from avelumab, atezolizumab, durvalumab, CX-072 (CytomX Therapeutics), BMS-936559 (MDX-1105, BMS);
(o′) LCL 161 in combination with an anti-CTLA-4 antibody selected from ipilimumab and tremelimumab;
(p′) LCL 161 in combination with an anti-TIGIT which is OMP-313M32 (mAb, OncoMed);
(q′) LCL 161 in combination with an anti-LAG-3 antibody selected from BMS-986016 (mAb Bristol Myers), LAG525 (mAb, Novartis), MGD013 (mAb, Macro-genics), REGN3767 (mAb, Regeneron Pharma), TSR-033 (mAb, Tesaro), and INCAGN022385 (mAb, Incyte Corp.);
(r′) LCL 161 in combination with an anti-Tim-3 antibody selected from LY3321367 (mAb, Eli Lilly and Company), MBG453 (mAb, Novartis), and TSR-022 (mAb, Tesaro);
(s′) LCL 161 in combination with any other therapy suggested or recommended in the OARAC Guidelines for the Use of Antiretroviral Agents in adults and Adolescents Living with HIV and/or EACS European Guidelines for treatment of HIV-positive adults in Europe.

Further preferred combinations involve the IAP inhibitor CUDC-427:
(a″) CUDC-427 or a CUDC-427 analogue in combination with an anti-PD-1 antibody;
(b″) CUDC-427 or a CUDC-427 analogue in combination with an anti-PD-L1 antibody;
(c″) CUDC-427 or a CUDC-427 analogue in combination with an anti-CTLA-4 antibody;
(d″) CUDC-427 or a CUDC-427 analogue in combination with an anti-TIGIT antibody;
(e″) CUDC-427 or a CUDC-427 analogue in combination with an anti-LAG-3 antibody;
(f″) CUDC-427 or a CUDC-427 analogue or a Debio-1143 analogue in combination with an anti-Tim-3 antibody;
(g″) CUDC-427 in combination with an anti-PD-1 antibody;
(h″) CUDC-427 in combination with an anti-PD-L1 antibody;
(i″) CUDC-427 in combination with an wth anti-CTLA-4 antibody;
(j″) CUDC-427 in combination with an anti-TIGIT antibody;
(k″) CUDC-427 in combination with an anti-LAG-3 antibody;
(l″) CUDC-427 in combination with an anti-Tim-3 antibody;
(m″) CUDC-427 in combination with an anti-PD-1 antibody selected from pembrolizumab, nivolumab, AMP514 (Medi0680, Amplimmune), REGN2810 (Regeneron), spartalizumab, tislelizumab and pidilizumab;
(n″) CUDC-427 in combination with an anti-PD-L1 antibody selected from avelumab, atezolizumab, durvalumab, CX-072 (CytomX Therapeutics), BMS-936559 (MDX-1105, BMS);
(o″) CUDC-427 in combination with an anti-CTLA-4 antibody selected from ipilimumab and tremelimumab;
(p″) CUDC-427 in combination with an anti-TIGIT which is OMP-313M32 (mAb, OncoMed);
(q″) CUDC-427 in combination with an anti-LAG-3 antibody selected from BMS-986016 (mAb Bristol Myers), LAG525 (mAb, Novartis), MGD013 (mAb, Macro-genics), REGN3767 (mAb, Regeneron Pharma), TSR-033 (mAb, Tesaro), and INCAGN022385 (mAb, Incyte Corp.);
(r″) CUDC-427 in combination with an anti-Tim-3 antibody selected from LY3321367 (mAb, Eli Lilly and Company), MBG453 (mAb, Novartis), and TSR-022 (mAb, Tesaro);
(s″) CUDC-427 in combination with any other therapy suggested or recommended in the OARAC Guidelines for the Use of Antiretroviral Agents in adults and Adolescents Living with HIV and/or EACS European Guidelines for treatment of HIV-positive adults in Europe.

Further preferred combinations involve the IAP inhibitor Birinapant:
(a′″) Birinapant or a Birinapant analogue in combination with an anti-PD-1 antibody;
(b′″) Birinapant or a Birinapant analogue in combination with an anti-PD-L1 antibody;
(c′″) Birinapant or a Birinapant analogue in combination with an anti-CTLA-4 antibody;
(d′″) Birinapant or a Birinapant analogue in combination with an anti-TIGIT antibody;
(e′″) Birinapant or a Birinapant analogue in combination with an anti-LAG-3 antibody;
(f′″) Birinapant or a Birinapant analogue or a Debio-1143 analogue in combination with an with anti-Tim-3 antibody;
(g′″) Birinapant in combination with an anti-PD-1 antibody;
(h′″) Birinapant in combination with an anti-PD-L1 antibody;
(i′″) Birinapant in combination with an anti-CTLA-4 antibody;
(j′″) Birinapant in combination with an anti-TIGIT antibody;
(k′″) Birinapant in combination with an anti-LAG-3 antibody;
(l′″) Birinapant in combination with an anti-Tim-3 antibody;
(m′″)Birinapant in combination with an anti-PD-1 antibody selected from pembrolizumab, nivolumab, AMP514 (Medi0680, Amplimmune), REGN2810 (Regeneron), spartalizumab, tislelizumab and pidilizumab;
(n′″) Birinapant in combination with an anti-PD-L1 antibody selected from avelumab, atezolizumab, durvalumab, CX-072 (CytomX Therapeutics), BMS-936559 (MDX-1105, BMS);
(o′″) Birinapant in combination with an anti-CTLA-4 antibody selected from ipilimumab and tremelimumab;
(p′″) Birinapant in combination with an anti-TIGIT which is OMP-313M32 (mAb, OncoMed);

1(q′″) Birinapant in combination with an anti-LAG-3 antibody selected from BMS-986016 (mAb Bristol Myers), LAG525 (mAb, Novartis), MGD013 (mAb, Macro-genics), REGN3767 (mAb, Regeneron Pharma), TSR-033 (mAb, Tesaro), and INCAGN022385 (mAb, Incyte Corp.);
(r′″) Birinapant in combination with an anti-Tim-3 antibody selected from LY3321367 (mAb, Eli Lilly and Company), MBG453 (mAb, Novartis), and TSR-022 (mAb, Tesaro);
(s′″) Birinapant in combination with any other therapy suggested or recommended in the OARAC Guidelines for the Use of Antiretroviral Agents in adults and Adolescents Living with HIV and/or EACS European Guidelines for treatment of HIV-positive adults in Europe.

Any one of these specific combinations listed under items (a) to (s) may be combined with further drugs as described herein. Similarly, the indications provided herein regarding patients, administration modes, dosages, etc. apply, of course, also with respect to the specific combinations listed under items (a) to (s).

### Further Components of Combination Therapy

As noted above, the combination therapy of the present invention is advantageously combined with the standards of care treatment as in the guidelines, in particular with the well-established antiretroviral therapy (ART) or combination antiretroviral therapy (cART). These therapies involve administration of typically two or more drugs selected from the following classes:
Nucleoside reverse transcriptase inhibitors (NRTIs) such as Zidovudine (Retrovir, AZT), Didanosine (Videx, Videx EC, ddl), Stavudine (Zerit, d4T), Lamivudine (Epivir, 3TC), Abacavir (Ziagen, ABC), Tenofovir, especially in its prodrug forms (i.e. disoproxil and alafenamide forms), a nucleotide analog (Viread, Vemlidy);
Non-nucleoside reverse transcriptase inhibitors (NNRTIs) such as Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera);
Protease inhibitors (PIs) such as Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV);
Entry inhibitors such as Enfuvirtide (Fuzeon, ENF, T-20), Maraviroc (Selzentry or Celsentri, MVC);
HIV integrase inhibitors such as Raltegravir (Isentress, RAL), Elvitegravir (EVG, part of the combination Stribild), Dolutegravir (Tivicay, DTG).

Possible schemes of treatment involving the combination therapy of the present invention may be as follows:
1. ART for several weeks/months (e.g. any duration from 2 weeks to 12 months) up to a low plasma HIV-1 level or a plasma HIV-1 level below the detection limit of clinical assays (for instance <50 copies/mL), continuation of ART and addition of ICI+IAP inhibitor
2. ART for several weeks/months (e.g. any duration from 2 weeks to 12 months) up to a low plasma HIV-1 level or a plasma HIV-1 level below the detection of clinical assays (for instance <50 copies/mL), continuation of ART and addition of ICI+IAP inhibitor+LRA
3. ART for several weeks/months (e.g. any duration from 2 weeks to 12 months) up to a plasma HIV-1 level below the detection of clinical assays (for instance <50 copies/mL), discontinuation of ART and administration of ICI+IAP inhibitor
4. ART for several weeks/months (e.g. any duration from 2 weeks to 12 months) up to a plasma HIV-1 level below the detection of clinical assays (for instance <50 copies/mL), discontinuation of ART and administration of ICI+IAP inhibitor+LRA
5. ICI+IAP inhibitor (this scheme is preferably used in patients refractory to ART)
6. ICI+IAP inhibitor+LRA (wherein LRA may be added immediately or after plasma HIV-1 level fall below the detection of clinical assays (for instance <50 copies/mL; this scheme is also preferably used in patients refractory to ART)
with:
ART: being existing antiretroviral therapy
ICI: Immune Checkpoint Inhibitor
IAP inh: IAP inhibitor
LRA: Latency Reversing Agent: HDAC inhibitor, PKC agonist, disulfiram, etc

In some embodiments, the combination therapy of the present invention includes in addition to the above-mentioned essential ingredients ICI and IAP inhibitor at least one NRTI, at least one NNRTI and optionally further drugs of the other categories.

In some embodiments, the IAP inhibitor is incorporated into a single unit dosage form further containing one or more of the above-mentioned drugs for ART.

The combination therapy of the present invention may also be combined with one or more of the following therapeutic approaches (based for instance on the information discussed by G. Darcis et al. in Trends in Immunology, March 2017, Vol. 38, No. 3 http://dx.doi.org/10.1016/j.it.2016.12.003):
PKC modulators like Bryostatin;
RIG-I-inducers like Acitretin;
BCL-2 inhibitors like venetoclax, obatoclax;
P13K/Akt Inhibitors like copanlisib (BAY 80-6946), MK-2206, AZD5363, ARQ 751, TAS-117 or BAY 1125976;
HDAC inhibitors like romidepsin, vorinostat, panobinostat;
histone methylation inhibitors (HMTi) like chaetocin and BIX-01294;
nucleoside analog methylation inhibitors such as 5-aza-2′-deoxycytidine (5-AzadC, trade name Dacogen);
DNA methyltransferase inhibitors (DNMTi),
inhibitors of bromodomain and extraterminal (BET) domain proteins (BETi);
disulfiram;
derivatives of ingenol ester, in particular ingenol B and ingenol-3-angelate;
toll-like receptor agonists like MGN1703, GS-9620 and GS-986;
therapeutic vaccines,
broadly neutralizing antibodies may also be co-administered. These are antibodies capable of broadly neutralizing diverse strains of HIV-1 infection when administered passively, as discussed for instance in R. Kumar et al. in Ther Adv Vaccines Immunother. 2018 Oct. 12; 6(4):61-68. doi: 10.1177/2515135518800689.

### 5.6. Pharmaceutical Compositions and Kits

Any of the drug compounds described herein as suitable for use in the present invention may be administered separately or as part of a pharmaceutical composition. However, this possibility is of course restricted to those cases, wherein the different components of the combination are suitable for the same mode of administration. Debio 1143, for instance, is preferably administered orally. It is not appropriate to combine Debio 1143 with an immune checkpoint inhibitor that needs to be administered intravenously. The present invention thus contemplates in particular pharmaceutical compositions comprising drug compounds as described herein, which can be administered by the same route. These are for instance, pharmaceutical compositions comprising IAP inhibitors like Debio 1143, which are suitable for oral administration, and further comprising one or more drug compounds for ART therapy. The present invention also contemplates pharmaceutical compositions for intravenous administration, which comprise an immune checkpoint inhibitor together with an IAP inhibitor like birinapant, which is suitable for intravenous administration. The pharmaceutical compositions of the invention may furthermore contain instructions for use.

In view of the above-mentioned differences in administration routes, the present invention also provides kits, wherein the two or more drugs are provided in two or more separate pharmaceutical compositions, each of which being formulated for a different route of administration. The kits of the invention may furthermore contain instructions for use.

Some embodiments of the present invention pertain to pharmaceutical compositions comprising only one of the two essential components, i.e. only an IAP inhibitor or only an immune checkpoint inhibitor, but wherein said pharmaceutical compositions are provided with instructions for use in the treatment of HIV involving co-administration of the other essential component, i.e. immune checkpoint inhibitor or IAP inhibitor, as appropriate. Such instructions for use may be given in the form of a printed patient leaflet, product labelling, or the like, or by means of oral or written instructions of the treating physician. The grant of a marketing authorization for one of the two essential components for use in combination with the other essential component for the treatment of HIV may also be regarded as an embodiment of the present invention.

In some embodiments of the present invention, the pharmaceutical compositions described above, i.e. the pharmaceutical compositions comprising both essential components or the pharmaceutical compositions comprising only one of the essential components, may comprise further active agents or combinations of active agents. It is, for instance, contemplated to provide a pharmaceutical composition for use in the treatment of HIV, the treatment being in combination with an immune checkpoint inhibitor, wherein the pharmaceutical composition comprises Debio 1143 together with one or more drugs for ART.

### 5.7. HIV Treatment

### 5.7.1. Patient Characteristics

In principle, the combination therapies, drug combinations and treatment methods of the present invention are suitable for use in any patient infected with HIV. The patient can be either naïve or in virological failure after ART (for instance incomplete suppression: HIV-Viral Load >200 copies/mL at 6 months after starting therapy in persons not previously on ART or rebound: e.g. confirmed HIV-VL >50 copies/mL in persons with previously undetectable HIV-VL), as defined by European guidelines. The patient can also be on ART with stable low HIV-Viral-Loads.

The patient can have HIV infection associated with other diseases, such as cancer (such as Kaposi's sarcoma, lymphoma, etc.) or other co-infection (such as tuberculosis, cytomegalovirus, HBV, etc.). In some embodiments, the patient is a patient infected with HIV-1. In some advantageous embodiments, the patient's CD4+ T cells show an increased level of one or more immune checkpoint molecules selected from PD-1, CTLA-4, TIGIT and/or LAG-3 at their surfaces. In some other advantageous embodiments, the patient's CD8+ T cells show an increased level of Tim-3 immune checkpoint molecules at their surfaces.

The combination therapies of the present invention may be particularly suitable for HIV patients suffering also from Kaposi sarcoma or lymphoma. In this sub-population of patients, the combination therapies of the present invention may be used for treating/curing HIV, the combination therapies of the present invention may be used for treating/curing Kaposi sarcoma or lymphoma, or the combination therapies of the present invention may be used for treating/curing HIV and Kaposi sarcoma at the same time.

### 5.7.2. Administration Forms and Dosages

The combination therapies of the present invention are not restricted to any particular type of administration. Instead, it is advantageous to identify and select an appropriate administration form for each component of the combination therapy.

In some embodiments, the IAP inhibitor is Debio 1143. In some specific embodiments, Debio 1143 is administered orally. In some embodiments, the Debio 1143 is administered in capsular form or tablet form. In some embodiments, the Debio 1143 is administered orally as a capsule containing 75, 100, 125, 150, 175, 200, 225, 250, 300, 400, 500, 600, 700, 750, 800, 900, or 1000, 1500 or 2000 mg Debio 1143. In some embodiments, the Debio 1143 is administered orally as a tablet containing 75, 100, 125, 150, 175, 200, 225 or 250 mg Debio 1143.

The therapeutically effective amount of Debio 1143 is typically about 75 to about 250 mg per day. Preferably, the therapeutically effective amount of Debio 1143 is about 75-100, 75-125, 75-150, 75-175, 75-200, 75-225, 100-125, 100-150, 100-175, 100-200, 100-225, 125-150, 125-175, 125-200, 125-225, 150-175, 150-200, 150-225, 175-200, 175-225, 200-225, 200-300, 225-300, 300-400, 300-500, 400-500, 400-600, 500-600, 500-700, 600-700, 600-750, 700-750, 700-800, 750-800, 750-900, 800-900, 800-1000, 900-1000, 200-400, 200-600, 200-800, 200-1000, 400-600, 400-800, 400-1000, 600-800 or 600-1000 mg per day. In some embodiments, the therapeutically effective amount of Debio 1143 is about 75, 100, 125, 150, 175, 200, 225, 250, 300, 400, 500, 600, 700, 750, 800, 900, or 1000 mg per day.

In certain embodiments, the therapeutically effective amount of Debio 1143 is administered as one dose one time per day. In certain embodiments, the therapeutically effective amount of Debio 1143 is divided into multiple doses that are administered as multiple doses two, three, or four times per day.

In some embodiments, the Debio 1143 is administered daily for 10 consecutive days. In some embodiments, the Debio 1143 is administered once daily for 10 consecutive days. In some embodiments, the method of treatment comprises a 28 day cycle comprising administering the Debio 1143 for 10 consecutive days, followed by administering no Debio 1143 for 4 consecutive days. In other embodiments, Debio 1143 may be administered for multiple periods of n consecutive days, interrupted by m days of no administration, wherein n is independently selected from 1, 2, 3, 4, 5, 6, 7, 8 and 9 and m is independently selected from 1, 2, 3 and 4.

The above information applies to Debio 1143 analogues as well. If other IAP inhibitors are used, administration forms, dosages and dosage regimes can be derived from the literature or be determined by the skilled person relying on routine drug dosage finding procedures.

In some embodiments of the invention, an immune checkpoint inhibitor (e.g. anti-PD-1 antibody) is administered by the intravenous route.

In some embodiments, the dosing regimen will comprise administering the immune checkpoint inhibitor at a dose of about 1, 2, 3, 4, 5, 6, 7, 8, 9, 10, 11, 12, 13, 14, 15, 16, 17, 18, 19 or 20 mg/kg or at a fixed dose (i.e. 240 mg nivolumab every two weeks or 200 mg pembrolizumab every 3 weeks) at intervals of about 14 days (±2 days) or about 21 days (±2 days) or about 30 days (±2 days) throughout the course of treatment. In other embodiments that employ an immune checkpoint inhibitor in the combination therapy, the dosing regimen will comprise administering the immune checkpoint inhibitor at a dose of from about 0.005 mg/kg to about 10 mg/kg, with intra-patient dose escalation. In certain embodiments, the therapeutically effective amount of immune checkpoint inhibitor (e.g. anti-PD-1 antibody), or antigen-binding fragment thereof, is about 10 mg/kg. In some embodiments, the immune checkpoint inhibitor (e.g., anti-PD-1 antibody), antigen-binding fragment thereof, is administered intravenously. In some embodiments, the anti-PD-1 antibody is nivolumab or pembrolizumab. Suitable dosages and treatment schemes for nivolumab include all dosages and treatment schemes indicated for cancer treatment, such as i.v. infusions of 240 mg over 30 minutes every 2 weeks or 480 mg over 60 minutes every 4 weeks. Suitable dosages and treatment schemes for pembrolizumab include all dosages and treatment schemes indicated for cancer treatment, such as i.v. infusions of 200 mg over 30 minutes every 3 weeks.

In some embodiments, the ICI and especially nivolumab or pembrolizumab is administered once every two weeks. In some embodiments, the ICI and especially nivolumab or pembrolizumab is administered on days 1 and 15 of a 28-day cycle. In some embodiments, ICI and especially nivolumab or pembrolizumab is administered intravenously. In certain embodiments, the immune checkpoint inhibitor is administered intravenously for 20-80 minutes at a dose of about 1-10 mg/kg body weight every 1-4 weeks. In a more preferred embodiment, the dose will be 4-8 mg/kg body weight administered as 30 min to 1-hour intravenous infusions every 1-4 weeks. Given the variability of infusion pumps from site to site, a time window of minus 10 minutes and plus 20 minutes is permitted.

If the ICI is avelumab, the drug is preferably administered as an 10 mg/kg body weight 1-hour intravenous infusions every 2 weeks (Q2W). Pharmacokinetic studies demonstrated that the 10 mg/kg dose of avelumab achieves excellent receptor occupancy with a predictable pharmacokinetics profile (see e.g., Heery et al., 2015. Proc ASCO Annual Meeting: abstract 3055). This dose is well tolerated, and signs of antitumor activity, including durable responses, have been observed.

The ICI may be administered up to 3 days before or after the scheduled day of administration of each cycle due to administrative reasons.

In further embodiments of the invention, the immune checkpoint inhibitor is an anti-PD-1 antibody, which is administered such that each single dose contains an amount of 150 mg to 300 mg antibody and preferably 200 mg to 240 mg.

In further embodiments of the invention, the immune checkpoint inhibitor is an anti-CTLA-4 antibody, which is administered such that each single dose contains an amount of 5 mg to 300 mg antibody and preferably 10 mg to 200 mg. If the CTLA-4 inhibitor is ipilimumab (tradename Yervoy), the recommended administration regimen of YERVOY is 3 mg/kg administered intravenously over a 90-minute period every 3 weeks for a total of 4 doses.

Generally, if there are any doubts regarding the administration of immune checkpoint inhibitors in the context of the combination therapy of the present invention, it is advisable to rely on the instructions for dosage and administration frequency established for cancer treatment by means of the respective immune checkpoint inhibitor.

Regarding the relative points in time of administering the two essential components of the combination therapies of the present invention, there is no particular restriction. That is, the two components may be administered simultaneously, or one of the components may be administered before or after the other component, such that the two components are both administered within a time period of no more than 28 days, preferably no more than 10 days.

More specifically, each of the following options is possible in the context of the present invention:
The first dose of the IAP inhibitor is administered in advance of the first dose of the immune checkpoint inhibitor.
The first dose of the IAP inhibitor is administered after the first dose of the immune checkpoint inhibitor.
The first dose of the IAP inhibitor is administered at the same time as the first dose of the immune checkpoint inhibitor.

During the ongoing treatment, the relative timing of administration of the two essential components of the combination therapies of the present invention is determined by the selected administration frequency (and interruptions of administration, if any).

The overall duration of the treatment is not particularly restricted. It may take any time period between a single administration of each of the two essential components up to prolonged administration of the two components over many weeks, months or even years. This will mainly depend on the plasma HIV viral load. In preferred embodiments, the combination therapy of the present invention is administered over a time period of from 10 days to 90 days and more preferably from 14 days to 42 days and particularly from 14 days to 28 days in the case of combination with ART. If the two essential components are administered without ART, the duration of treatment could be up to 12 months.

### 6. EXAMPLES

### Example 1: HIV Reactivation Using Latent Reporter Cell Lines

The experiment analyzed the ability of DEB10-1143 at reactivating latent HIV using three well-characterized latent T cell lines—JLat 10.6-GFP, 2D10 and 5A8 (Jordan A, Bisgrove D, Verdin E (2003) EMBO J 22(8): 1868-1877. doi: 10.1093/emboj/cdg188. PMID: 12682019; Sakane N, Kwon HS, Pagans S, Kaehlcke K, Mizusawa Y, Kamada M, et al. (2011) PLoS Pathog 7(8): e1002184. PMID: 21876670; Pearson R, Kim Y K, Hokello J, Lassen K, Friedman J, Tyagi M, et al. (2008) J Virol 82(24): 12291-12303. PMID: 18829756). These cell lines contain part of the HIV genome and the GFP reporter gene and are unable to produce infective HIV particles. Drug titration and kinetic studies were conducted. Debio-1143 was tested alone or in combination with the current antiretroviral therapy (ART), consisting of 20 μM tenofovir disoproxil fumarate [TDF], 10 μM emtricitabine [FTC] and 1 μM raltegravir [RAL]. ART and DEB10-1143 were added simultaneously to cells.

Method: Cells (100,000 cells/100 μL) were treated in triplicate for 0 and 72 h with increasing concentrations of DEB10-1143 (0 to 20.5 μM) used alone or in combination with ART (see above). ART and DEB10-1143 were added simultaneously to cells. Cells were analyzed for GFP expression by flow cytometry at the indicated time points counting all cells per replicate, and reported as % GFP+cells.

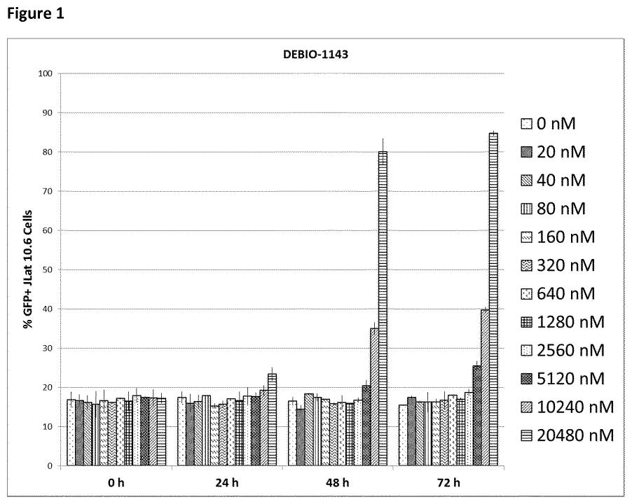

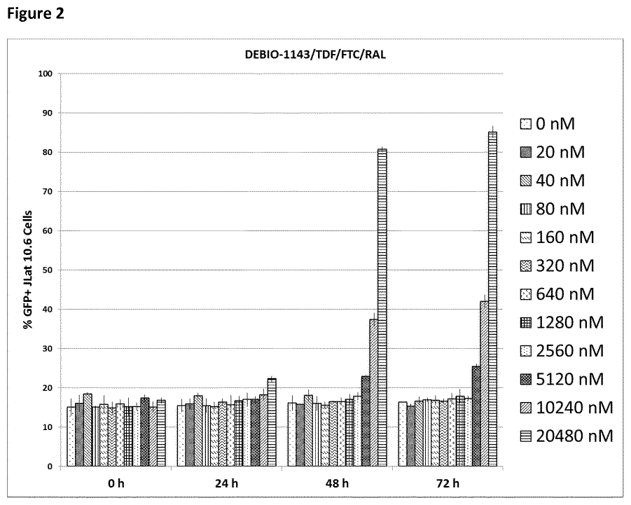

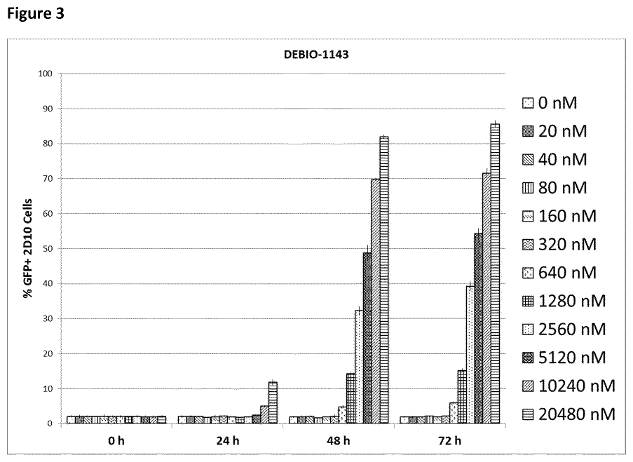

Results and conclusion: The results of the experiments are shown graphically in FIG. 1 to FIG. 6. It is apparent that DEB10-1143 as a single agent possesses the capacity of activating latent HIV in latent GFP reporter cell lines. The degree of reactivation depends on the cell line used and is drug concentration dependent. ART does not influence the degree of DEB10-1143 latency reversal, and ART alone is known to not cause HIV reactivation in these cell lines (ART alone was not tested here). The latent HIV cell line 2D10 displays a low GFP background in the absence of stimulus compared to the two other cell lines and shows the best degree of HIV reactivation by DEB10-1143. Altogether these data indicate that Debio 1143 is a strong latency reversal agent and can be combined with ART treatment which is required to prevent new rounds of infection.

### Example 2: Cellular Cytotoxic Analysis of DEB10-1143

This experiment examined the cellular cytotoxicity of DEB10-1143 alone and DEB10-1143+ART on the human CD4+ T cell line JLat 10.6. DEB10-1143 was tested as well on human primary CD4+ T lymphocytes. Drug titration and kinetic studies were conducted.

Method: Cells (100,000 cells/100 μL) were treated in triplicate for 72 h with increasing concentrations of DEB10-1143 (0 to 20 μM) used alone or in combination with ART (20 μM TDF, 10 μM FTC and 1 μM Raltegravir). ART and DEB10-1143 were added simultaneously to cells. Saponin (0.5%) was used as positive control due to its ability to induce cell death.

Cellular cytotoxicity was analyzed by Lactate dehydrogenase (LDH) assay and CellTiter Glo assay.

LDH assay: LDH is an oxidoreductase enzyme that catalyses the interconversion of pyruvate and lactate. Cells release LDH into the bloodstream after tissue damage or red blood cell hemolysis. Since LDH is a fairly stable enzyme, it has been widely used to evaluate the presence of damage and toxicity of tissue and cells. In this particular assay, LDH reduces NAD to NADH, which is specifically detected by colorimetric (490 nm) assay.

CellTiter Glo assay: The CellTiter-Glo® 2.0 Assay determines the number of viable cells in culture by quantifying ATP, which indicates the presence of metabolically active cells. Luminescence readout is directly proportional to the number of viable cells in culture. Data are expressed in luminescence (RLU) in triplicate

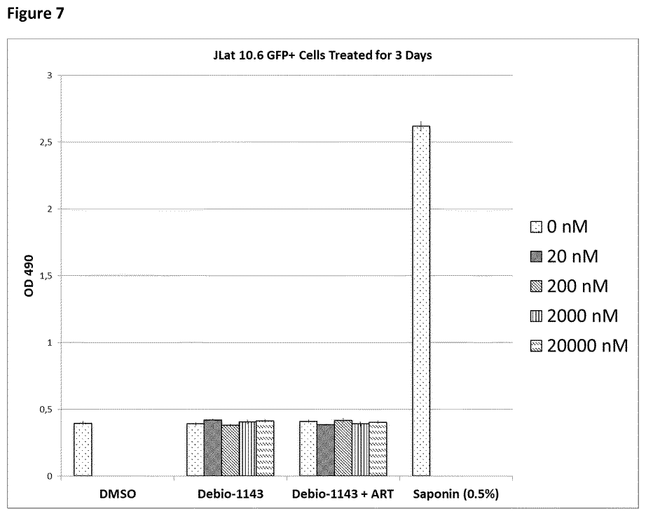

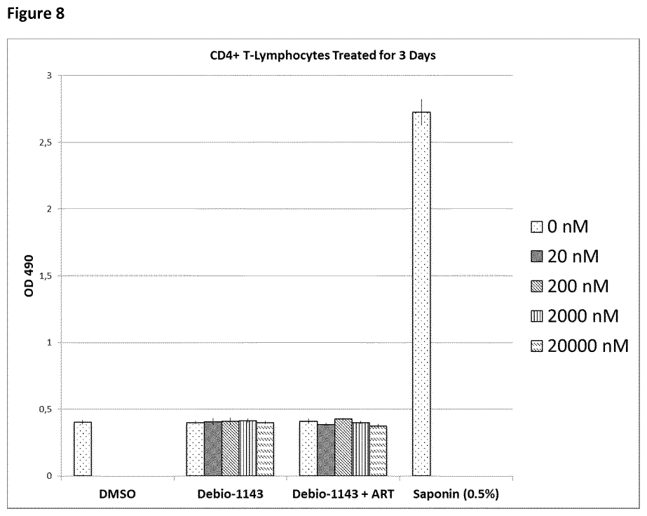

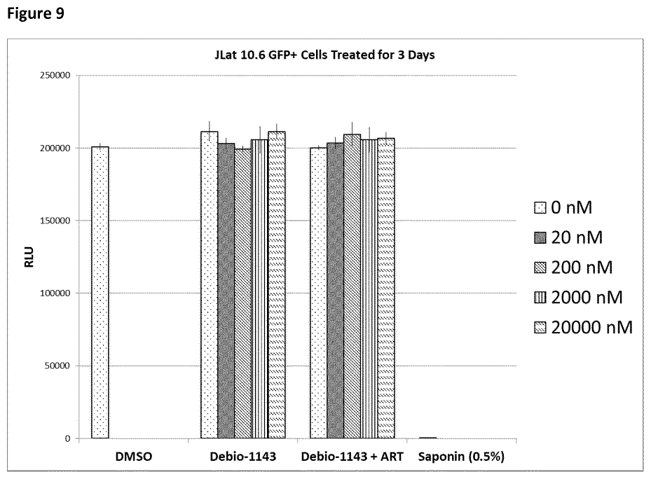

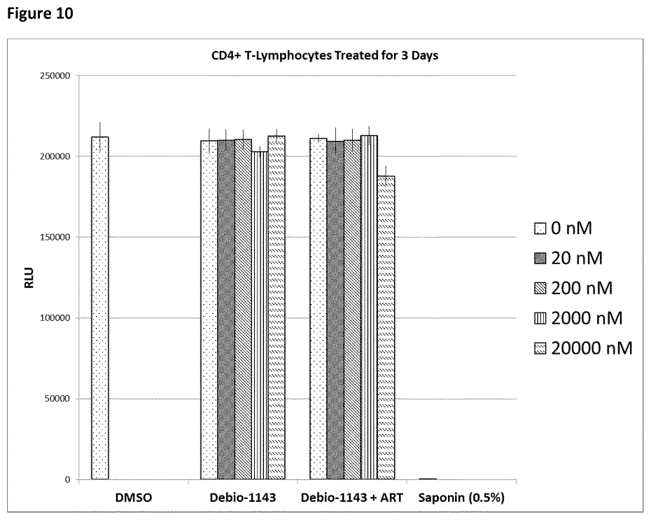

Results and conclusions: The results of the experiments are shown graphically in FIG. 7 to FIG. 10. It was found that DEB10-1143, even at high concentration (20 μM), presents the advantage of exhibiting no significant cytotoxicity on the CD4+ T cell line JLat 10.6, as well as on primary CD4+ T-lymphocytes.

### Example 3: HIV Reactivation in Resting CD4+ T-Lymphocytes Derived from ART-Treated HIV Patients

The experiments of this example analyzed the ability of DEB10-1143 at reactivating latent HIV in resting CD4+ T-lymphocytes isolated from peripheral blood monocytic cells (PBMCs) derived from HIV patients currently under ART. Drug titration and kinetic studies were conducted. Debio-1143 was tested alone or in combination with ART.

Method: Resting CD4+ T cells were isolated using the EasySep™ Human Resting CD4+ T Cell Isolation Kit, which is designed to isolate resting CD4+ T cells from fresh or previously frozen PBMCs by immunomagnetic negative selection. The EasySep™ procedure involves labeling unwanted cells with antibody complexes and magnetic particles. The magnetically labeled cells are separated from the untouched desired cells by using an EasySep™ magnet and simply pouring or pipetting the desired cells into a new tube. Isolated resting CD4+ T cells (50,000 cells/50 μL) were treated in duplicate for 0, 24, 48 and 72 h with increasing concentrations of DEB10-1143 (0 to 20.5 μM) used alone or in combination with ART (20 μM TDF, 10 μM FTC and 1 μM Raltegravir). Amounts of virions released from cells were quantified at the indicated time points by quantifying HIV RNA levels (HIV RNA copies/mL) in supernatant of cell culture using one-step reverse transcriptase quantitative real-time PCR (ABI custom TaqMan Assays-by-Design) according to the manufacturer's instructions. Primers were 5-CATGTTTTCAGCATTATCAGAAGGA-3 and 5-TGCTTGATGTCCCCCCACT-3, and MGB probe 5-FAM-CCACCCCACAAGATTTAAACACCATGCTAA-Q-3, where FAM is 6-carboxyfluorescein.

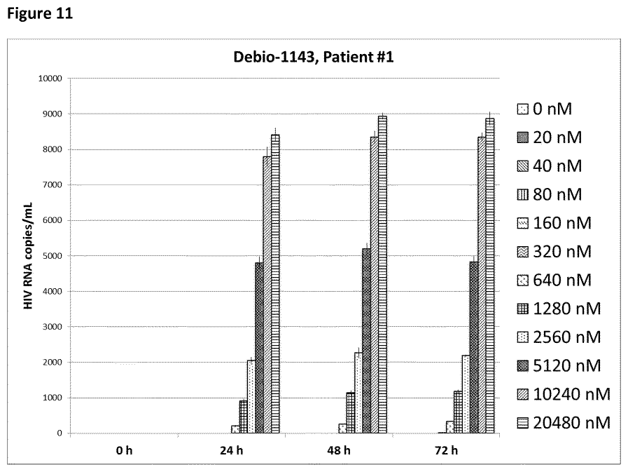

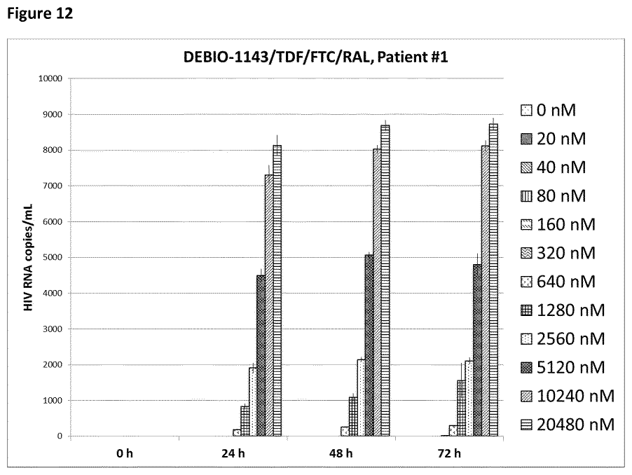

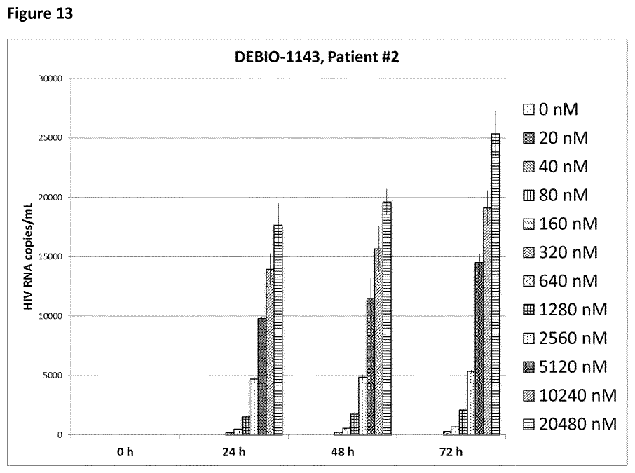

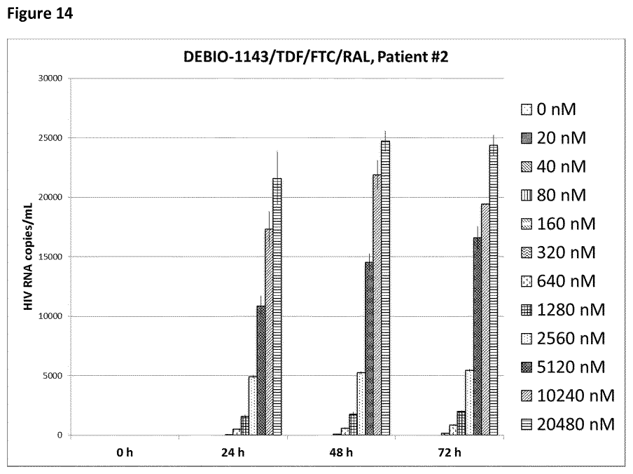

Results and conclusion: Results are shown in FIG. 11 to FIG. 14. Remarkably, DEB10-1143 possesses the capacity of reactivating latent HIV in resting CD4+ T cells isolated from PBMCs derived from two HIV patients currently undergoing ART treatment. The reactivation was drug dose-dependent and already maximal after 24 h of DEB10-1143 treatment. ART did not influence the degree of DEB10-1143 latency reversal. This indicates that Debio 1143 has the potential to exert its beneficial latency reversal effect not only in reporter cell lines, but also in a more physiological context in resting CD4+ T cells from individuals on ART treatment, which is required to prevent new rounds of infection.

### Example 4: Safety and Efficacy Study of Debio-1143 Alone or in Combination with an Anti-Programmed Death 1 (anti-PD-1) Antibody in a HIV-1 Latency Model in Humanized BLT Mice

### 1. Creation of Humanized Bone Marrow/Liver/Thymus (BLT) Mice

Methods: Humanized BLT mice were generated by implanting human fetal liver and thymus tissues under the kidney capsule of an immunodeficient NOD scid gamma (NSG=NOD.Cg-Prkdc^{scid}II12rg^{tm1Wjl}/SzJ) mouse, followed by an administration of autologous human fetal liver CD34+ cells (human hematopoietic stem cells). In BLT mice, T cell education occured in the human thymic tissue, resulting in complete systemic reconstitution of all major human hematopoietic lineages including T, B, monocyte/macrophage, dendritic, and natural killer cells. The extensive systemic and genital mucosal reconstitution with human lymphoid cells rendered female humanized BLT mice susceptible to both vaginal and rectal HIV infection.

Humanized BLT mice were generated by implanting 1-mm³ pieces of human fetal liver and thymus tissues (Advanced Bioscience Resources) under the kidney capsule in 6 to 8-week-old female NSG mice (Jackson Laboratories) bred at The Scripps Research Institute (TSRI). Each cohort was produced with tissues from a single donor. CD34+ HSPC were purified from autologous fetal liver tissue, isolated by magnetic bead selection for CD34+ cells (Miltenyi), phenotyped cytometrically for engraftment success (CD34+, HLA DR-), and cryopreserved until injection (200,000 CD34+ cells) into mice 3 weeks after thymus/liver implantation. Human reconstitution was verified by flow cytometry (CD45+, CD3+, CD4+ and CD8+) in peripheral blood of the mice.

R5 HIV (JR-CSF) was produced by 293T transfection and quantified by p24 ELISA. Humanized mice were infected with JR-CSF (200 ng of p24) and viral replication quantified weekly by qPCR for up to 16 weeks.

### 2. Tolerability Pre-Study

Methods: Debio-1143 (100 mg/kg; QD (daily) 1-5, p.o. (orally)) together with anti-PD-1 antibody (Bio X Cell; 200 μg/dose, BIW (biweekly), i.p. (intraperitoneally)) were administered to 3 HIV-1 infected BLT mice. Body weight, HIV-1 loads in blood (qPCR analysis) and PD-1 expression on CD8+ T cells (PBMCs isolation, antibody staining and flow cytometry analysis) were analyzed at day 0 before Debio-1143 and anti-PD1 antibody administration and day 7.

Results and conclusion: The analysis revealed that combination treatment for 1 week was well tolerated by HIV-1-infected BLT mice. No clinical signs of toxicity or body weight loss were observed on treatment or thereafter.

### 3. Efficacy Study

Methods: Thirty-two BLT mice were infected with HIV-1. 12 weeks later mice were split in 4 groups (n=8), treated with various regimen and analyzed for 6 weeks. Group A received both vehicles for 4 weeks. Group B received Debio-1143 (100 mg/kg; QD1-5, p.o.) for 4 weeks (Debio-1143 was given 5 days a week for 4 weeks). Group C received the anti-PD-1 antibody (200 μg/dose, i.p., BIW) for 4 weeks. Group D received the combination of Debio-1143 (100 mg/kg; QD1-5, p.o.) together with the anti-PD-1 antibody (200 μg/dose i.p. BIW) for 4 weeks. Body weight, HIV-1 loads and PD-1 expression on CD8+ T cells were analyzed once weekly. After the 4 weeks of treatment, three mice per group were sacrificed for bioanalytical assessments. The remaining mice (4×5=20) were sacrificed 2 weeks after treatment completion and viral RNA levels in organ reservoirs (spleen, thymic organoid, lung, spleen, lymph nodes and liver) were quantified by qPCR.

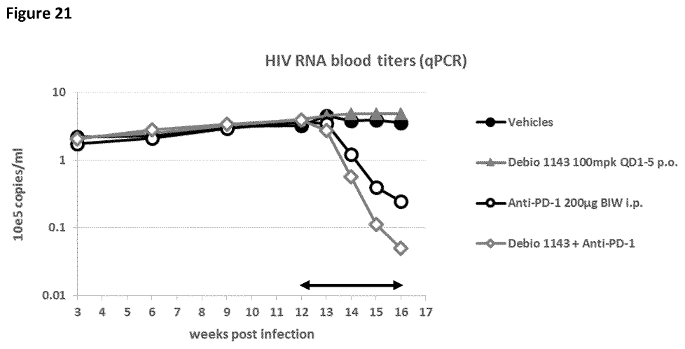

Results and conclusion: Anti-PD-1 alone reduced the HIV titers in blood—but low virus levels remained detectable in all individuals. Debio 1143 alone increased viral titers (potentially suggesting latency-reversal as found previously in vitro); the combination of Anti-PD-1 with Debio 1143 further reduced viral blood titers with 5 out of 8 mice having no detectable levels after 4 weeks of treatment (FIG. 21). Similar results were observed at the end of the 2 week post-treatment observation, where reduced but detectable HIV blood titers were found in 5 out of 5 animals of the anti-PD-1 group, and further reduced titers in the combination group (with 2 out of 5 animals without detectable virus).

CD8+ T cells play an important role in controlling viral infections including HIV; however, chronic infection leads to expression of co-inhibitory immune checkpoint molecules (such as PD-1) on CD8+ T cells and their functional exhaustion as marked by lower proliferation, cytokine production, and cytotoxic abilities. This provides a rationale for using immune checkpoint inhibitors in HIV infection to stimulate a sustained antiviral immune response. Of note, the BLT mouse model used here does recapitulate this functional T cell exhaustion characterized by PD-1 expression, and the published kinetics of PD-1 expression in CD8+ T cells informed the experimental design to allow for manifestation of T cell exhaustion for 12 weeks after infection, ensuring that anti-PD-1 treatment was started in a relevant context. (D. M. Brainard et al., JOURNAL OF VIROLOGY, July 2009, p. 7305-7321, doi:10.1128/JVI.02207-08).

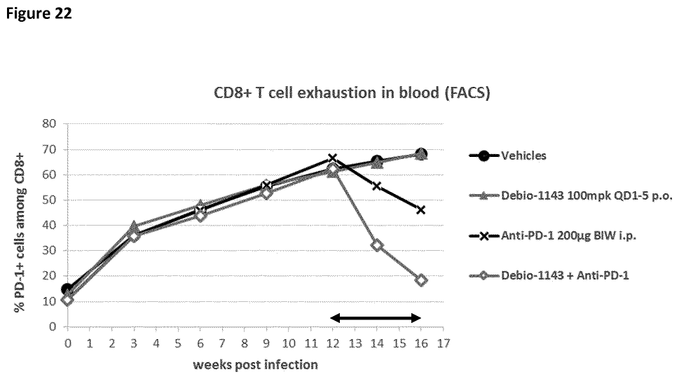

At 12 weeks post-infection using flow cytometry T cell exhaustion was confirmed prior to treatment start and results were in line with published data that roughly 60% of CD8+ T cells expressed PD-1 at that time point (D.M. Brainard et al., JOURNAL OF VIROLOGY, July 2009, p. 7305-7321, doi:10.1128/JVI.02207-08). Assessment of PD-1 expression in CD8+ T cells after 4 weeks of treatment showed that while vehicle treatment had no impact, anti-PD-1 alone reduced the % of exhausted CD8+ T cells in circulation to 46%. Surprisingly, the combination of Anti-PD-1 with Debio 1143 greatly enhanced this effect, synergistically reducing the fraction of PD-1 expressing CD8+ T cells to only 18% (FIG. 22). These results suggest that the combination has a positive effect on the activation status of the immune cell population in charge of fighting the HIV infection.

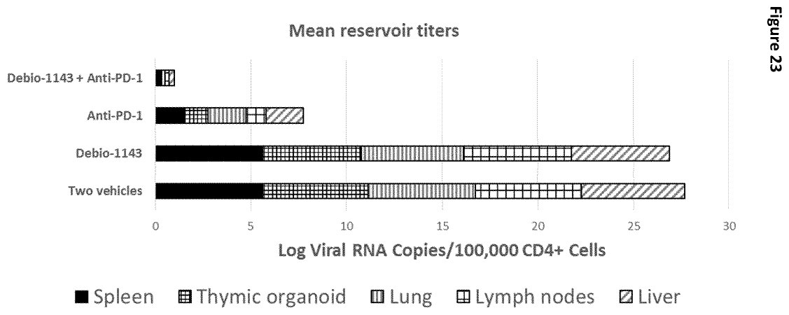

Importantly, 2 weeks after treatment completion PCR quantification of viral RNA levels in organ reservoirs (CD4+ cells in: spleen, thymic organoid, lung, spleen, lymph nodes and liver) showed that Debio 1143 alone has no effect whereas anti-PD-1 alone reduces the HIV load in organs, but the combination has a much greater effect on the HIV organ titers across all individuals and organs (FIG. 23). Surprisingly, in contrast to Anti-PD-1 alone, the combination was able to render some organs in some individuals entirely virus-free (Table 1). This finding strongly suggests that using the combination treatment in an optimized dose schedule, it may be possible to achieve elimination of the virus from all organs of a treated individual, constituting a cure of the HIV infection.

TABLE 1
Tissue HIV Load at Week 18 displayed as
Log Viral RNA Copies/100,000 CD4+ Cells.

| | Mouse | | Thymic | | Lymph | |
|---|---|---|---|---|---|---|
| Treatment | # | Spleen | organoid | Lung | nodes | Liver |
| Vehicles | 1 | 5.7 | 5.5 | 5.5 | 6 | 5.8 |
| | 2 | 4.6 | 5.1 | 5.8 | 4.6 | 5.3 |
| | 3 | 6.1 | 5.8 | 6.3 | 6.1 | 6 |
| | 4 | 5.5 | 4.9 | 6 | 5.4 | 5.2 |
| | 5 | 6.3 | 6.1 | 4.4 | 5.7 | 4.6 |
| | mean | 5.64 | 5.48 | 5.6 | 5.56 | 5.38 |
| | SD | 0.472 | 0.384 | 0.52 | 0.448 | 0.416 |
| Debio-1143 | 1 | 4.4 | 4.6 | 3.9 | 4.4 | 5.2 |
| | 2 | 6.4 | 5.3 | 5.7 | 5 | 4.2 |
| | 3 | 5.2 | 3.9 | 4.4 | 5.3 | 4.9 |
| | 4 | 5.5 | 5.7 | 6.1 | 6.3 | 5.5 |
| | 5 | 6.7 | 6.2 | 6.5 | 7.2 | 5.9 |
| | mean | 5.64 | 5.14 | 5.32 | 5.64 | 5.14 |
| | SD | 0.728 | 0.712 | 0.936 | 0.888 | 0.472 |
| Anti-PD-1 | 1 | 0.6 | 0.4 | 2.3 | 1.1 | 2.3 |
| | 2 | 1.1 | 0.7 | 1.4 | 0.9 | 1.8 |
| | 3 | 3.3 | 2.4 | 3.1 | 1.2 | 2 |
| | 4 | 0.4 | 0.9 | 1.7 | 0.7 | 1.5 |
| | 5 | 2.4 | 1.6 | 1.4 | 1.4 | 2.2 |
| | mean | 1.56 | 1.2 | 1.98 | 1.06 | 1.96 |
| | SD | 1.032 | 0.64 | 0.576 | 0.208 | 0.248 |
| Debio-1143 + | 1 | 0.3 | 0.1 | 0.2 | 0.3 | 0.4 |
| Anti-PD-1 | 2 | 0.1 | 0 | 0.1 | 0 | 0.1 |
| | 3 | 0.2 | 0.3 | 0.2 | 0.4 | 0.3 |
| | 4 | 0.1 | 0 | 0.1 | 0.1 | 0.1 |
| | 5 | 0.2 | 0.3 | 0.3 | 0.3 | 0.4 |
| | mean | 0.18 | 0.14 | 0.18 | 0.22 | 0.26 |
| | SD | 0.064 | 0.128 | 0.064 | 0.136 | 0.128 |

### Example 5: In Vitro HIV-1 Reactivation Potency and Cytotoxicity of Debio 1143, or Different Latency-Reactivating Agents (LRAs) Alone or in Combination

We compared the efficacy of Debio-1143 at reversing HIV-1 latency with that of other LRAs. We then analyzed the cytotoxicity of Debio-1143 with that of the other LRAs on 2D10 cells. Drug titration was conducted.

Methods: HIV-1 latent GFP-reporter 2D10 cells (100,000 cells/100 μL) were treated in triplicate for 48 h with increasing concentrations of either only Debio-1143 or the LRAs (2000 nM or 20000 nM), or with Debio 1143 (2000 nM or 20000 nM) in combination with the LRAs at a single predetermined concentration (see Table 2). HIV reactivation was analyzed via GFP expression by flow cytometry.

For analysis of cell viability, cells (100,000 cells/100 μL) were treated in triplicate for 48 h with increasing concentrations of Debio-1143 or other known LRAs (0 to 20 μM). Cellular cytotoxicity was analyzed by Lactate dehydrogenase (LDH) assay. LDH is an oxidoreductase enzyme that catalyzes the interconversion of pyruvate and lactate. Cells release LDH into the bloodstream after tissue damage or red blood cell hemolysis. Since LDH is a fairly stable enzyme, it has been widely used to evaluate the presence of damage and toxicity of tissue and cells. In this particular, LDH reduces NAD to NADH, which is specifically detected by colorimetric (490 nm) assay.

TABLE 2
LRA concentrations used in combination with Debio 1143.

| | | Concentration |
|---|---|---|
| LRA | Class | used [nM] |
| panobinostat | pan-HDAC | 2 |
| entinostat | HDAC class | 1000 |
| | I-specific | |
| vorinostat | HDAC class | 1000 |
| | I, II and IV | |
| chaetocin | DMTs | 100 |
| PH02 | Unknown | 2000 |
| | (Hashemi et al., 2017) | |
| bryostatin-1 | PKC | 500 |
| JQ1 | BRDs | 1000 |

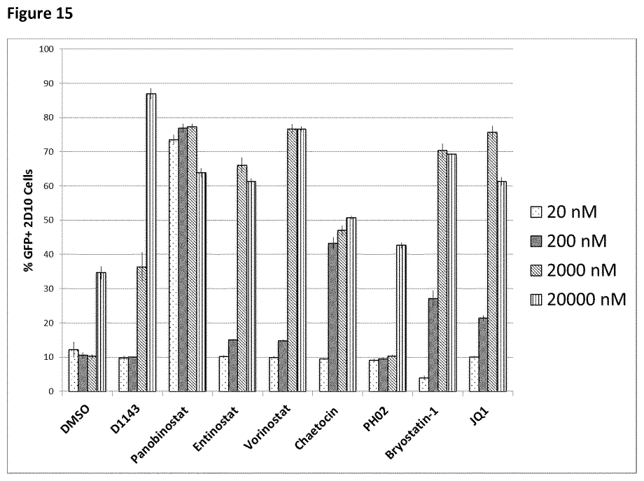

Results and conclusion: Debio-1143 as well as all other LRAs tested as single agents reversed HIV-1 latency in GFP reporter 2D10 cells. DMSO also displayed HIV reactivation at the highest concentration tested. We found that Debio-1143 is one of the most potent LRA. The results of this experiment are shown in FIG. 15.

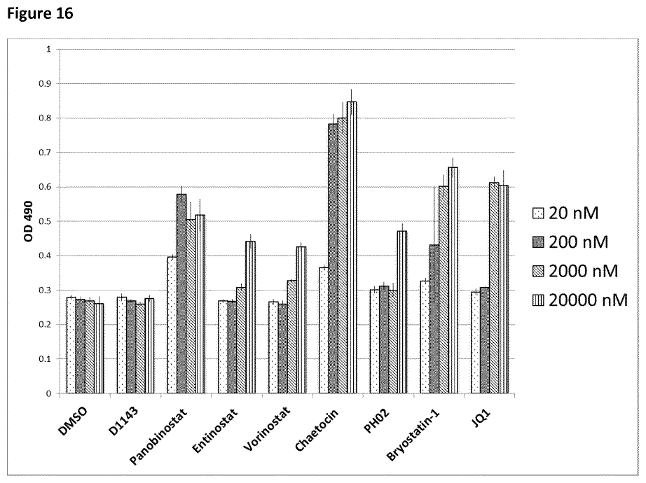

It was furthermore found that Debio-1143, even at high concentration (20 μM), did not exhibit any cytotoxicity. In contrast, some of the other LRAs were highly toxic including panobinostat, chaetocin, bryostatin-1 and JQ1. The results of this experiment are shown in FIG. 16.

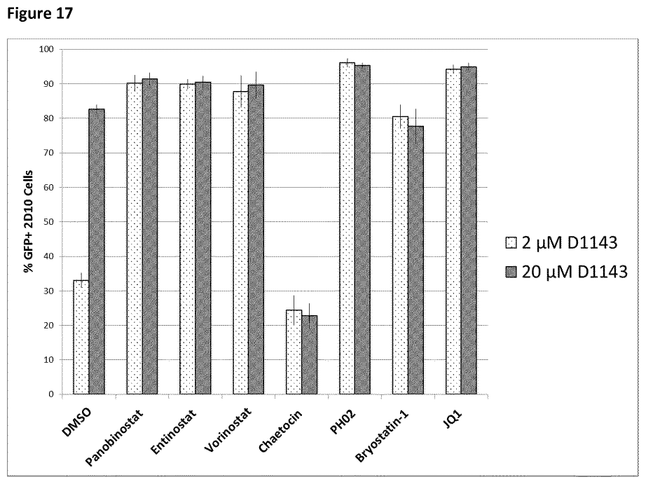

As regards the combination of Debio 1143 with other LRAs (Panobinostat, Entinostat, Vorinostat, Chaetocin, PH01, Bryostatin-1 and JQ-1), the degree of reactivation was concentration dependent for all tested LRAs. Except for DMSO at the lowest tested concentration and Chaetocin at both tested concentrations, all drug combinations displayed high degrees of HIV reactivation already at the 2 μM Debio 1143 concentration. The results of this experiment are shown in FIG. 17.

### Example 6: cIAP1 Degradation and NF-κB Modulation in HIV-1 Latent 2D10 Cell Line

It was investigated whether Debio-1143 reverses HIV-1 latency by acting on the NF-κB pathway. Canonical and non-canonical NF-κB signaling pathways play an important role in the reactivation of latent HIV-1, implicating its regulation as an important therapeutic strategy for latency reversal. We asked whether Debio-1143 activates the HIV-1 long terminal repeat (LTR) via the noncanonical NF-κB signaling since Debio-1143 has been shown to bind to and degrade the negative regulator of the noncanonical NF-κB signaling—BIRC2.

Method: 2D10 (A) or 293T (B) and CD4+ T cells (C) cells were treated with DMSO, Debio-1143 (1 μM) and TNFα (10 ng/ml) for the indicated times and analyzed by Western blotting for BIRC2 and IkBα protein expression using antibodies specifically detecting human BIRC2, human IkBα, or human CypA, such as commercially available (e.g. from R&D Systems, Cell Signaling, Santa Cruz Biotechnology, or other sources).

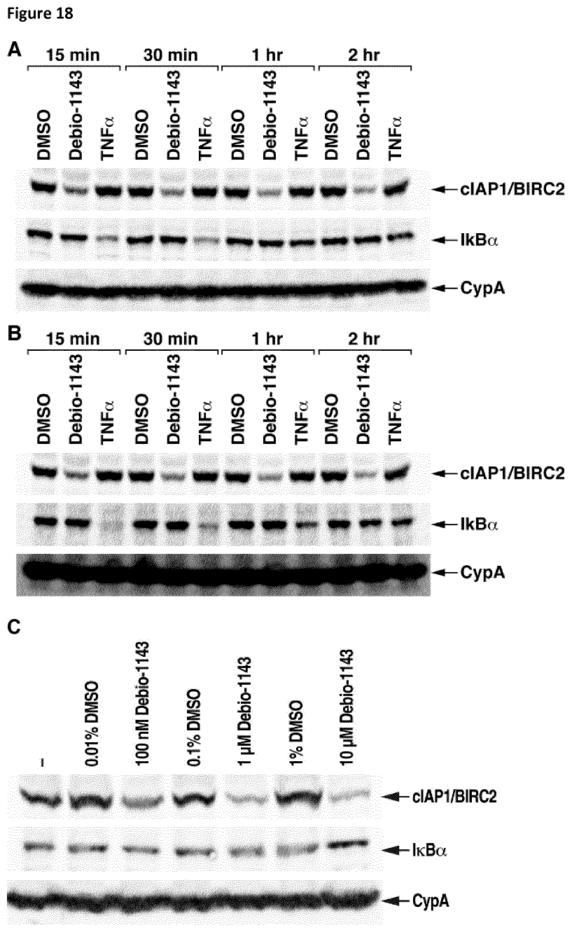

Results and conclusion: The results are shown in FIG. 18. Debio-1143, but not TNFα triggers rapid (<15 min) BIRC2 degradation in both 2D10 and 293T cells. In contrast, TNFα triggers IkBα degradation with a rebound after 2 h, while Debio-1143 has no effect (A, B).

This is in accordance with the notion that lkBa degradation is a hallmark of the canonical NF-κB signaling activation. BIRC2 degradation was analyzed in CD4+ T cells and found to be dose-dependent (C). Similar levels of cyclophilin A (CypA) indicate that similar amounts of cell lysates were analyzed.

These data strongly suggest that Debio-1143 activates the noncanonical NF-κB signaling by triggering rapid degradation of BIRC2—negative regulator of the noncanonical NF-κB signaling—leading to i) activation of the HIV-1 LTR; ii) viral transcription; and iii) reversal of HIV-1 latency.

### Example 7: In Vitro the Killing Effect of Debio-1143 on the Lysis of HIV-Infected Resting CD4+ T Cells (rCD4) by CD8+ T Cells and NK Cells

It was investigated whether the Debio-1143-induced reversal of HIV-1 latency, by restoring HIV-1 protein production in rCD4, would permit viral peptide exposure with MHC on infected rCD4+ T cells and allow recognition and lysis by CD8+ T cells and/or NK cells.

Method: Human CD4+ T cells, CD8+ T cells and NK cells were isolated following manufacturer's instruction (beads coated with specific antibodies) from one blood donor (500 mL). Ninety percent of CD4+ T cells were infected with HIV-1 (JR-CSF) (1 μg of p24), incubated for 4 days to allow establishment of infection, and then treated with ART for 3 days. Suppression of replication was verified by p24 ELISA in supernatants. rCD4 were then isolated following manufacturer's instruction. Infected and ART-treated rCD4 were mixed with uninfected CD4+ T cells, CD8+ T cells and NK cells a 1:1 ratio (100,000 cells) for 24 and 48 in triplicate. Mono-culture of each cell populations were used as controls. DMSO or Debio-1143 (0, 1 and 10 μM) were added to mono- or co-cultures for 24 and 48 h. Cell killing was quantified by LDH activity.

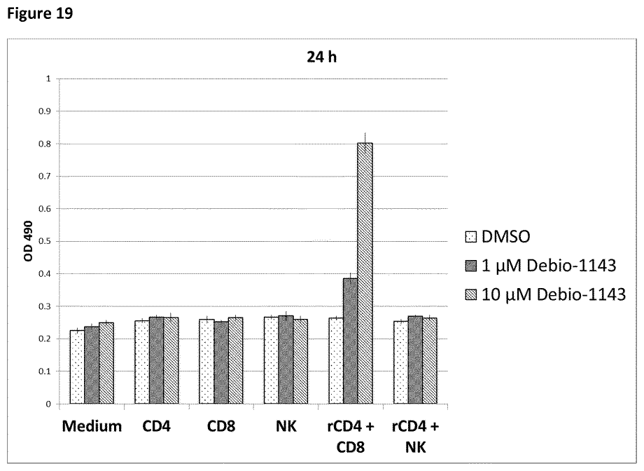

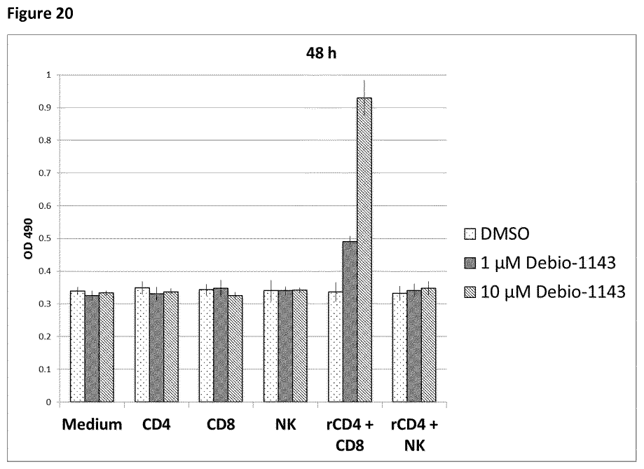

Results and conclusion: Low cell death was observed in all mono- and co-cultures except for the rCD4:CD8+ T cells co-culture, suggesting that Debio-1143 triggers viral peptide exposure on infected rCD4 allowing their recognition and lysis by CD8+ T cells. These results are shown in FIG. 19 and FIG. 20.

### Example 8: In Vitro Assays of Latency Reversal Effect of Further Combinations

### Example 8.1: In Vitro HIV-1 Reactivation Potency of IAPa, ICI, or IAPa/ICI Combinations in Ex Vivo Co-Cultures of rCD4+ and CD8+ Cells from ART-Treated HIV POatients

The efficacy of IAPa, ICI or IAPa/ICI combinations in reversing HIV-1 latency was tested in HIV-1 infected rCD4+ cells co-cultured for 48 h with CD8+ cells from the same donor.

Method: Blood from one HIV-1-infected patient under ART was collected (500 mL) and CD4+ and CD8+ cells isolated using beads coated with specific antibodies following manufacturer's instruction (MojoSort™ Human Cell Isolation Kits from Biolegend). The CD4+ T cells were re-infected with HIV-1 (JR-CSF) (1 μg of p24), incubated for 3 days to allow establishment of infection, and treated with ART (FTC 150 mg/kg+TDF 150 mg/kg+Raltegravir 80 mg/kg) for 7 days to ensure presence of enough latent HIV CD4+ cells (rCD4+) in the culture. Upon ART removal, rCD4 were then isolated following manufacturer's instruction (EasySep™ Human Resting CD4+ T Cell Isolation Kit). The isolated rCD4 were mixed with autologous (from the same patient) uninfected CD8+ T cells at a 1:1 ratio (100,000 cells) and incubated in duplicate for 48 h with different IAPa (μM) or ICI (10 μg/ml) in monotherapy, or the IAPa/ICI combinations. Amounts of virions released from cells were quantified at the indicated time points by quantifying HIV RNA levels (HIV RNA copies/mL) in supernatant of cell culture using one-step reverse transcriptase quantitative real-time PCR (ABI custom TaqMan Assays-by-Design) according to the manufacturer's instructions. Primers were 5-CATGTTTTCAGCATTATCAGAAGGA-3 and 5-GCTTGATGTCCCCCCACT-3, and MGB probe 5-FAM-CCACCCCACAAGATTTAAACACCATGCTAA-Q-3, where FAM is 6-carboxyfluorescein.

The following agents were tested in this experiment:

### Immune Checkpoint Inhibitors

### Anti-PD-L1

### Agent: Invitrogen CD274 (PD-L1, B7-H1) Monoclonal Antibody (MIH1)

Source: eBioscience
https://www.thermofisher.com/antibody/product/CD274 PD L1 B7 H1 Antibody-clone-MIH1-Monoclona1/14-5983-80
Reference: Tian, X. et al. The upregulation of LAG 3 on T cells defines a subpopulation with functional exhaustion and correlates with disease progression in HIV infected subjects. J. Immunol. 194,3873-3882 (2015). https://www.jimmunol.org/content/194/8/3873.1ong

### Anti-CTLA4

### Agent: Purified Mouse Anti-Human CD152, Clone BNI3

### Source: BD Biosciences

http://www.bdbiosciences.com/us/applications/research/t-cell-immunology/regulatory-t-cells/surface-markers/human/purified-mouse-anti-human-cd152-bni3/p/555851
Reference: Kaufmann, D. E. et al. Upregulation of CTLA 4 by HIV-specific CD4+ T cells correlates with disease progression and defines a reversible immune dysfunction. Nat. Immunol. 8,1246-1254 (2007).

### Anti-TIGIT

Agent: Ultra-LEAF™ Purified anti-human TIGIT (VSTM3) Antibody, clone A15153G

### Source: BioLegend

https://www.biolegend.com/en-us/products/ultra-leaf-purified-anti-human-tigit-vstm3-antibody-14287
Reference: Chew, G. M. et al. TIGIT marks exhausted T cells, correlates with disease progression, and serves as a target for immune restoration in HIV and SIV Infection. PloS Pathog. 12, e1005349 (2016).

### Anti-TIM3

Agent: Purified anti-human CD366 (Tim-3) Antibody, a-TIM-3 (F38-2E2; 345003)

### Source: BioLegend

https://www.biolegend.com/en-us/search-results/purified-anti-human-cd366-tim-3-antibody-6119
Reference: Lichtenegger FS, Rothe M, Schnorfeil FM, et al. Targeting LAG-3 and PD-1 to Enhance T Cell Activation by Antigen-Presenting Cells. Front Immunol. 2018; 9:385. Published 2018 Feb. 27. Doi:10.3389/fimmu.2018.00385
https://www.ncbi.nlm.nih.gov/pmc/articles/PMC5835137/#B21

### IAP inhibitors

### Agent: Debio 1143

Source: Synthesis in-house according to published literature procedure (Example 16 of WO 2008/128171)

### Agent: LCL 161

### Source: MedChem Express HY-15518

https://www.medchemexpress.com/LCL161.html?src=google-product&gclid=Cj0KCQiwk_qBRD8ARIsAOteukCztQzDsXbOSAgx7Ui2I4IrkVv_2fb4HZzkKUVm vGw-hemIJKb9V80aAsDvEALw_wcB

### Agent: CUDC-427

### Source: MedChem Express HY-15835

https://www.medchemexpress.com/CUDC-427.html

### Agent: Birinapant

### Source: Medchem Express HY-16591

https://www.medchemexpress.com/Birinapant.html

Results and conclusion: The results are summarized in Tables 3 and 4 below. It may be derived from these tables that the tested combinations show a significantly increased efficacy in reversing HIV latency.

TABLE 3

| | | 1 μM | 1 μM | 1 μM |
|---|---|---|---|---|
| | no IAPa | D1143 | LCL161 | CUDC-427 |
| 0.1% DMSO | 0* | 3 | 3 | 3 |
| Control IgG2 | 0* | 3 | 3 | 3 |
| Control IgG1 | 0* | 3 | 2 | 3 |
| Anti-PD-L1 | 6** | 13 | 12 | 6 |
| Anti-CTLA4 | 16 | 24 | 25 | 20 |
| Anti-TIGIT | 6 | 14 | 12 | 13 |
| Anti-TIM3 | 6 | 14 | 13 | 13 |

*no RNA copies detectable in the 3 control conditions
**All other RNA levels were detected and classified into different latency reversal classes. Up to 500 RNA copies per ml were classified as “0”. Between 501 and 1000 RNA copies/ml were classified as “1”. Subsequent classes were defined accordingly in steps of 500 RNA copies/ml.

TABLE 4

| | | 1 μM |
|---|---|---|
| | no IAPa | Birinapant |
| 0.1% DMSO | 0* | 3 |
| Control IgG2 | 0* | 3 |
| Control IgG1 | 0* | 3 |
| Anti-PD-L1 | 5** | 12 |
| Anti-CTLA4 | 14 | 24 |
| Anti-TIGIT | 5 | 14 |
| Anti-TIM3 | 6 | 14 |

*no RNA copies detectable in the 3 control conditions
**All other RNA levels were detected and classified into different latency reversal classes. Up to 500 RNA copies per ml were classified as “0”. Between 501 and 1000 RNA copies/ml were classified as “1”. Subsequent classes were defined accordingly in steps of 500 RNA copies/ml.

### Example 8.2: In Vitro HIV-1 Reactivation Potency of IAPa, ICI, or IAPa/ICI Combinations in Ex Vivo Treated PBMCs from Three ART-Treated HIV Patients

The experiments of this example analyzed the ability of IAPa, ICIs, or IAPa/ICI combinations at reactivating latent HIV in resting CD4+ T-lymphocytes isolated from peripheral blood monocytic cells (PBMCs) derived from HIV patients currently under ART.

Method: Resting CD4+ T cells were isolated from PBMCs of three ART-treated HIV patients by negative selection and magnetic separation (Easysep Mouse/Human Chimera Isolation Kits from STEMCELL technologies). Isolated resting CD4+ T cells (50,000 cells/50 μL) were treated in duplicate for 48h with different IAPa (1 μM) or ICI (10 μg/ml) in monotherapy, or the IAPa/ICI combinations. Amounts of virions released from cells were quantified at the indicated time points by quantifying HIV RNA levels (HIV RNA copies/mL) in supernatant of cell culture using one-step reverse transcriptase quantitative real-time PCR (ABI custom TaqMan Assays-by-Design) according to the manufacturer's instructions. Primers were 5-CATGTTTTCAGCATTATCAGAAGGA-3 and 5-GCTTGATGTCCCCCCACT-3, and MGB probe 5-FAM-CCACCCCACAAGATTTAAACACCATGCTAA-Q-3, where FAM is 6-carboxyfluorescein.

The experiment was carried out using the same agents as described above for Example 8.1 (with the exception that no birinapant was used).

Results and conclusion: The results of this assay are summarized in Table 5 below. They show the same trend of significantly increased levels of HIV latency reversal for the drug combinations of the present invention.

TABLE 5

| | | 1 μM | 1 μM | 1 μM |
|---|---|---|---|---|
| | no IAPa | D1143 | LCL161 | CUDC-427 |
| 0.1% DMSO | 0* | 1 | 1 | 1 |
| Control IgG2 | 0* | 1 | 1 | 1 |
| Control IgG1 | 0* | 1 | 1 | 1 |
| Anti-PD-L1 | 2** | 6 | 6 | 3 |
| Anti-CTLA4 | 8 | 11 | 12 | 10 |
| Anti-TIGIT | 2 | 7 | 6 | 6 |
| Anti-TIM3 | 3 | 7 | 6 | 6 |

*no RNA copies detectable in the 3 control conditions
**All other RNA levels were detected and classified into different latency reversal classes. Up to 500 RNA copies per ml were classified as “0”. Between 501 and 1000 RNA copies/ml were classified as “1” Subsequent classes were defined accordingly in steps of 500 RNA copies/ml.

### Example 8.3: Ex Vivo HIV-1 Reactivation Potency of IAPa, ICI, or IAPa/ICI Combinations in PBMCs from ART-Treated BLT Mice

The efficacy of IAPa, ICI or IAPa/ICI combinations in reversing HIV-1 latency was tested in resting CD4+ T cells derived from ART-treated HIV-1-infected humanized BLT mice.

Method: BLT mice were infected with JR-CSF (200 ng of p24) and treated daily with ART (FTC 150 mg/kg+TDF 150 mg/kg+Raltegravir 80 mg/kg) until HIV-1 RNA levels were drastically reduced. Human resting CD4+ T cells from ten mice were isolated from blood, thymic organoid, lung, spleen, bone marrow, lymph nodes and liver. Isolated human resting CD4+ T cells were pooled, counted, split and treated in duplicate for 48 h with different IAPa (1 μM) or ICI (10 μg/ml) in monotherapy, or the IAPa/ICI combinations. Amounts of virions released from cells were quantified at the indicated time points by quantifying HIV RNA levels (HIV RNA copies/mL) in supernatant of cell culture using one-step reverse transcriptase quantitative real-time PCR (ABI custom TaqMan Assays-by-Design) according to the manufacturer's instructions. Primers were 5-CATGTTTTCAGCATTATCAGAAGGA-3 and 5-GCTTGATGTCCCCCCACT-3, and MGB probe 5-FAM-CCACCCCACAAGATTTAAACACCATGCTAA-Q-3, where FAM is 6-carboxyfluorescein.

The experiment was carried out using the same agents as described above for Example 8.1 (with the exception that no birinapant was used).

Results and conclusion: The results are summarized in Table 6 below. The data derived from HIV-1-infected cells isolated from an in vivo mouse therapeutic setting are consistent with (and thus confirm) the data derived from cells isolated from HIV-1-infected human patients provided in Examples 8.1 and 8.2 above. There is a pronounced increase in the HIV latency reversal effect when combining an IAP inhibitor with an ICI.

TABLE 6

| | | 1 μM | 1 μM | 1 μM |
|---|---|---|---|---|
| | no IAPa | D1143 | LCL161 | CUDC-427 |
| 0.1% DMSO | 0* | 2 | 2 | 2 |
| Control IgG2 | 0* | 2 | 2 | 2 |
| Control IgG1 | 0* | 2 | 2 | 2 |
| Anti-PD-L1 | 3** | 6 | 6 | 3 |
| Anti-CTLA4 | 7 | 10 | 11 | 10 |
| Anti-TIGIT | 3 | 6 | 6 | 6 |
| Anti-TIM3 | 3 | 7 | 6 | 6 |

*no RNA copies detectable in the 3 control conditions
**All other RNA levels were detected and classified into different latency reversal classes. Zero RNA copies per ml were classified as “0”. Up to 2500 RNA copies per ml were classified as “1”. Between 2501 and 5000 RNA copies/ml were classified as “2”. Subsequent classes were defined accordingly in steps of 2500 RNA copies/ml.

Using different concentrations of the active agents may allow to achieve more pronounced effects, especially when using active agents other than those employed in the experiments described hereinabove.

1.-18. (canceled)

19. A pharmaceutical composition comprising an IAP inhibitor and an immune checkpoint inhibitor, wherein the IAP inhibitor is preferably selected from Debio 1143, Debio 1143 analogues, LCL-161, TL-32711/birinapant, CUDC 427/GDC 0917, APG-1387, ASTX660, SBP-0636457, JP1201, AZD5582, and BI 891065 and the immune checkpoint inhibitor is preferably selected from the group consisting of CTLA-4 antagonists, PD-1 inhibitors, LAG-3 inhibitors, TIGIT inhibitors, Tim-3 inhibitors and PDL-1 inhibitors.

20. The pharmaceutical composition according to claim 19, wherein the IAP inhibitor is Debio-1143.

21. The pharmaceutical composition for use according to claim 19, wherein the immune checkpoint inhibitor is an inhibitor of PD-1 or PD-L1.

22. The pharmaceutical composition according to claim 20, wherein the immune checkpoint inhibitor is selected from the group consisting of pembrolizumab, nivolumab, spartalizumab, tislelizumab, pidilizumab, AMP514, REGN2810, AMP-224, spartalizumab, avelumab, atezolizumab , CX-072, BMS-936559, MPDL3280A, MED14736, CA-170 and durvalumab.

23. The pharmaceutical composition according to claim 21, wherein the immune checkpoint inhibitor is pembrolizumab or nivolumab.

24. (canceled)

25. (canceled)

26. The pharmaceutical composition according to claim 19, comprising one or more further drugs selected from the group consisting of NRTIs such as Zidovudine (Retrovir, AZT), Didanosine (Videx, Videx EC, ddl), Stavudine (Zerit, d4T), Lamivudine (Epivir, 3TC), Abacavir (Ziagen, ABC), Tenofovir, a nucleotide analog (Viread, TDF); NNRTIs such as Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera); integrase inhibitors such as Raltegravir (Isentress, RAL), Elvitegravir (EVG, part of the combination Stribild), Dolutegravir (Tivicay, DTG); protease inhibitors such as Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV); entry inhibitors such as Enfuvirtide (Fuzeon, ENF, T-20), Maraviroc (Selzentry or Celsentri, MVC); PKC modulators like Bryostatin; RIG-I-inducers like Acitretin; BCL-2 inhibitors like venetoclax, obatoclax; Pl3K/Akt Inhibitors like copanlisib (BAY 80-6946), MK-2206, AZD5363, ARQ 751, TAS-117 or BAY 1125976; HDAC inhibitors like romidepsin, vorinostat, panobinostat; histone methylation inhibitors (HMTi) like chaetocin and BIX-01294; nucleoside analog methylation inhibitors such as 5-aza-2′-deoxycytidine (5-AzadC, trade name Dacogen); DNA methyltransferase inhibitors (DNMTi), inhibitors of bromodomain and extraterminal (BET) domain proteins (BETi); disulfiram; derivatives of ingenol ester, in particular ingenol B and ingenol-3-angelate; toll-like receptor agonists like MGN1703, GS-9620 and GS-986; therapeutic vaccines, and broadly neutralizing antibodies.

27. A method of treatment of HIV infection in a patient in need thereof, said method comprising administering to the patient the IAP inhibitor together with an immune checkpoint inhibitor, wherein the IAP inhibitor is preferably selected from Debio 1143, LCL-161, TL-32711/birinapant, CUDC 427/GDC 0917, APG-1387, ASTX660, SBP-0636457, JP1201, AZD5582, and BI 891065 and the immune checkpoint inhibitor is preferably selected from the group consisting of CTLA-4 antagonists, PD-1 inhibitors, LAG-3 inhibitors, TIGIT inhibitors, Tim-3 inhibitors and PDL-1 inhibitors.

28. The method according to claim 27, wherein the IAP inhibitor is Debio-1143.

29. The method according to claim 27 or 28, wherein the immune checkpoint inhibitor is an inhibitor of PD-1 or PD-L1.

30. The method according to claim 29, wherein the immune checkpoint inhibitor is selected from the group consisting of pembrolizumab, nivolumab, spartalizumab, tislelizumab, pidilizumab, AMP514, REGN2810, AMP-224, spartalizumab, avelumab, atezolizumab , CX-072, BMS-936559, MPDL3280A, MED14736, CA-170 and durvalumab.

31. The method according to claim 30, wherein the immune checkpoint inhibitor is pembrolizumab or nivolumab.

32. The method according to claim 27, wherein the IAP inhibitor is administered to the patient prior to, simultaneously with, or after administration of the immune checkpoint inhibitor.

33. The method according to claim 27, wherein the method of treatment leads to stimulation of CD8+ T cells.

34. The method according to claim 27, wherein the method reverses viral latency.

35. The method according to claim 27, wherein the method involves administration of one or more further drugs selected from the group consisting of NRTIs such as Zidovudine (Retrovir, AZT), Didanosine (Videx, Videx EC, ddl), Stavudine (Zerit, d4T), Lamivudine (Epivir, 3TC), Abacavir (Ziagen, ABC), Tenofovir, a nucleotide analog (Viread, TDF); NNRTIs such as Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera); integrase inhibitors such as Raltegravir (Isentress, RAL), Elvitegravir (EVG, part of the combination Stribild), Dolutegravir (Tivicay, DTG); protease inhibitors such as Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV); entry inhibitors such as Enfuvirtide (Fuzeon, ENF, T-20), Maraviroc (Selzentry or Celsentri, MVC); PKC modulators like Bryostatin; RIG-I-inducers like Acitretin; BCL-2 inhibitors like venetoclax, obatoclax; PI3K/Akt Inhibitors like copanlisib (BAY 80-6946), MK-2206, AZD5363, ARQ 751, TAS-117 or BAY 1125976; HDAC inhibitors like romidepsin, vorinostat, panobinostat; histone methylation inhibitors (HMTi) like chaetocin and BIX-01294; nucleoside analog methylation inhibitors such as 5-aza-2′-deoxycytidine (5-AzadC, trade name Dacogen); DNA methyltransferase inhibitors (DNMTi), inhibitors of bromodomain and extraterminal (BET) domain proteins (BETi); disulfiram; derivatives of ingenol ester, in particular ingenol B and ingenol-3-angelate; toll-like receptor agonists like MGN1703, GS-9620 and GS-986; therapeutic vaccines, and broadly neutralizing antibodies.

36. The method according to claim 28, wherein the immune checkpoint inhibitor is an inhibitor of PD-1 or PD-L1.

37. The method according to claim 28, wherein the immune checkpoint inhibitor is selected from the group consisting of pembrolizumab, nivolumab, spartalizumab, tislelizumab, pidilizumab, AMP514, REGN2810, AMP-224, spartalizumab, avelumab, atezolizumab , CX-072, BMS-936559, MPDL3280A, MEDl4736, CA-170 and durvalumab.

38. The method according to claim 37, wherein the immune checkpoint inhibitor is pembrolizumab or nivolumab.

39. The method according to claim 28, wherein the method involves administration of one or more further drugs selected from the group consisting of NRTIs such as Zidovudine (Retrovir, AZT), Didanosine (Videx, Videx EC, ddl), Stavudine (Zerit, d4T), Lamivudine (Epivir, 3TC), Abacavir (Ziagen, ABC), Tenofovir, a nucleotide analog (Viread, TDF); NNRTIs such as Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera); integrase inhibitors such as Raltegravir (Isentress, RAL), Elvitegravir (EVG, part of the combination Stribild), Dolutegravir (Tivicay, DTG); protease inhibitors such as Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV); entry inhibitors such as Enfuvirtide (Fuzeon, ENF, T-20), Maraviroc (Selzentry or Celsentri, MVC); PKC modulators like Bryostatin; RIG-I-inducers like Acitretin; BCL-2 inhibitors like venetoclax, obatoclax; PI3K/Akt Inhibitors like copanlisib (BAY 80-6946), MK-2206, AZD5363, ARQ 751, TAS-117 or BAY 1125976; HDAC inhibitors like romidepsin, vorinostat, panobinostat; histone methylation inhibitors (HMTi) like chaetocin and BIX-01294; nucleoside analog methylation inhibitors such as 5-aza-2′-deoxycytidine (5-AzadC, trade name Dacogen); DNA methyltransferase inhibitors (DNMTi), inhibitors of bromodomain and extraterminal (BET) domain proteins (BETi); disulfiram; derivatives of ingenol ester, in particular ingenol B and ingenol-3-angelate; toll-like receptor agonists like MGN1703, GS-9620 and GS-986; therapeutic vaccines, and broadly neutralizing antibodies.

40. The pharmaceutical composition according to claim 20, wherein the immune checkpoint inhibitor is an inhibitor of PD-1 or PD-L1.


SEQUENCE LISTING (4 sequences)

SEQ ID NO: 1 - DNA, 25 bp, Artificial Sequence
  Primer
catgttttcagcattatcagaagga

SEQ ID NO: 2 - DNA, 19 bp, Artificial Sequence
  Primer
tgcttgatgtccccccact

SEQ ID NO: 3 - DNA, 30 bp, Artificial Sequence
  MGB probe
  misc_feature (1)..(1): 6-carboxyfluorescein
  misc_feature (30)..(30): MGB quencher
ccaccccacaagatttaaacaccatgctaa

SEQ ID NO: 4 - DNA, 18 bp, Artificial Sequence
  Primer
gcttgatgtccccccact